(19) United States
(12) Patent Application Publication
(10) Pub. No.: US 2023/0381115 A1
(43) Pub. Date: Nov. 30, 2023

(54) Close-packed fibrous dosage form

(71) Applicant: Aron H. Blaesi, Cambridge, MA (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 18/129,026

(22) Filed: Mar. 30, 2023

Related U.S. Application Data
(63) Continuation of application No. PCT/US2021/053033, filed on Sep. 30, 2021.
(60) Provisional application No. 63/085,893, filed on Sep. 30, 2020.
(60) Provisional application No. 63/158,870, filed on Mar. 9, 2021.
(60) Provisional application No. 63/229,016, filed on Aug. 3, 2021.

(51) Int. Cl. A61K 9/70; A61K 47/38; A61K 45/06

(52) U.S. Cl. CPC A61K 9/7007; A61K 47/38; A61K 45/06

(57) ABSTRACT

Herein a dosage form comprising a three-dimensional structural framework of repeatably arranged, close-packed fibrous structural elements is presented. The elements include active ingredient particles, an excipient matrix to bind and carry said particles, and pores. The disclosed dosage forms may, for example, be applied for delivery of large doses of drug into the blood stream.

### CROSS-REFERENCE TO RELATED INVENTIONS

This application is a continuation of, and incorporates herein by reference in its entirety, the International Application No. PCT/US2021/053033 filed on Sep. 30, 2021 and titled “Close-packed fibrous dosage form”, which claims priority to and the benefit of, the U.S. Provisional Application No. 63/085,893 filed on Sep. 30, 2020, the U.S. Provisional Application No. 63/158,870 filed on Mar. 9, 2021, and the U.S. Provisional Application No. 63/229,016 filed on Aug. 3, 2021. All foregoing applications are hereby incorporated by reference in their entirety.

This application is related to and incorporates herein by reference in their entirety, the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, the U.S. application Ser. No. 16/860,911 filed on Apr. 28, 2020 and titled “Expandable structured dosage form for immediate drug delivery”, the U.S. application Ser. No. 16/916,208 filed on Jun. 30, 2020 and titled “Dosage form comprising structural framework of two-dimensional elements”, the U.S. application Ser. No. 17/237,034 filed on Apr. 21, 2021 and titled “Method for 3D-micro-patterning”, the U.S. application Ser. No. 17/327,721 filed on May 23, 2021 and titled “Expandable multi-excipient dosage form”, the International Application No. PCT/US19/19004 filed on Feb. 21, 2019 and titled “Expanding structured dosage form”, the International Application No. PCT/US19/52030 filed on Sep. 19, 2019 and titled “Dosage form comprising structured solid-solution framework of sparingly-soluble drug and method for manufacture thereof”, the International Application No. PCT/US21/22857 filed on Mar. 17, 2021 and titled “Expandable, multi-excipient structured dosage form for prolonged drug release”, and the International Application No. PCT/US21/22860 filed on Mar. 17, 2021 and titled “Method and apparatus for 3D-micro-patterning”.

### BACKGROUND OF THE INVENTION

At present, the immediate-release solid dosage forms generally comprise drug and excipient granules that are compacted into a porous tablet. Upon immersion in an aqueous medium, the medium may percolate the open pores and interdiffuse with the water-soluble excipient. The inter-granular bonds may then be severed, the granules may be released into the surrounding medium, and the drug may dissolve.

However, because the pores in the compacted dosage forms may not be well connected, fluid percolation may not be uniform; hence numerous excipients (˜5-10) and multiple statistical process steps may be required to ensure that the dosage form meets the specifications. As a result, dosage form development and manufacture may be resource-intensive and time-consuming. Moreover, addition of such common excipients as lactose, starch, sugars, polyols, and so on, is undesirable because they may cause allergies or other adverse effects in some patients. For further details related to the specifications of solid dosage forms, or oral solid dosage forms in particular, see, e.g., Y. Qiu, L. Lirong, G. Zhang, Y. Chen, W. Porter, Developing oral solid dosage forms: pharmaceutical theory and practice, Academic Press, Burlington, M A, 2008; or M. E. Aulton, K. M. G. Taylor, Aulton's Pharmaceutics: The Design and Manufacture of Medicines, fourth edn, Churchill Livingstone Elsevier, Oxford, U K, 2013. For further details related to the manufacture of solid dosage forms, see, e.g., F. Muzzio, T. Shinbrot, B. J. Glasser, Powder technology in the pharmaceutical industry: the need to catch up fast, Powder Technol. 124 (2002) 1-7. For further details related to adverse effects of present excipient formulations, see, e.g., G. Pifferi, P. Restani, The safety of pharmaceutical excipients, Il Farmaco 58 (2003) 541-550; or D. Reker, S. M. Blum, C. Steiger, K. E. Anger, J. M. Sommer, J. Fanikos, G. Traverso, “Inactive” ingredients in oral medications, Sci. Trans. Med. 11 (2019) 6753.

The above limitations could be mitigated by dosage forms with controlled microstructure and contiguous void space, such as the 3D-micro-patterned fibrous dosage forms the present inventors (Blaesi and Saka) have introduced recently (e.g., in the U.S. patent application Ser. No. 15/482,776). It was shown that if the inter-fiber spacing and excipient weight fraction are large, and the drug dose is small, the fibrous forms can be deterministically manufactured by 3D-printing. Moreover, rapid drug release can be achieved even with a single, biologically inert excipient.

Many drug therapies, however, require large drug doses. Therefore, the dosage forms must also be manufacturable, and the release rate controllable, if the fibers are densely packed and the drug weight fraction in the fibers is large.

In this specification, accordingly, concepts for the manufacture and design of dosage forms with close-packed fibers and high drug loading in the fibers are disclosed. The disclosed concepts are not limited to fibrous dosage forms and may be applicable to structured (e.g., 3D-printed, 3D-micro-patterned, etc.) dosage forms in general.

### DESIGN CONSIDERATIONS

The following design considerations and any supporting illustrations will enable one of ordinary skill in the art to more readily understand the invention presented throughout this disclosure. They are not meant to be limiting in any way.

FIG. 1 schematically illustrates a non-limiting method for preparing dosage forms with close-packed fibers and high drug loading in the fibers as disclosed herein. The initial material may be a particulate mixture 107 of a large weight fraction of sparingly water-soluble drug particles 130 and a small weight fraction of a highly water-soluble polymeric excipient 110, FIG. 1a. By adding a substantial quantity of solvent 115 (e.g., water) to the mixture 107, the excipient 130 may be plasticized and a viscous suspension 108 of the plasticized excipient 116 and the drug particles 130 may be formed. The suspension 106 may then be extruded through a syringe needle 145, patterned as a fibrous dosage form 103, and dried.

During drying, as solvent (water) evaporates the wet fibers 105 (and the wet dosage form) may initially shrink uniformly, FIGS. 1b and 1c. Eventually, however, the drug particles 130 may touch each other and further shrinkage of the particulate suspension 106 may be blocked. As water may continue to evaporate past that point, capillaries and pores 140 may develop in the fibers 107 to compensate for the lost water volume, FIG. 1d.

Thus, as shown schematically in the non-limiting FIG. 2a, after drying the solid dosage form 200 may comprise a three dimensional structural framework of densely-packed, repeatably arranged fibers 210 surrounded by contiguous free space 220. The fiber 210 microstructure may comprise a small weight fraction of excipient 240 forming an excipient 240 matrix through the fiber 210 thickness, a large weight fraction of drug particles 230, and pores 245. By introducing pores 245 into the microstructure of the solid fibers 210, manufacture of fibrous dosage forms 200 with very high drug 230 loading (e.g., with very large volume fraction of drug particles 230, or with very large drug 230 weight fraction, etc.) may be feasible.

Upon immersing the dry, solid dosage form 200 in a dissolution fluid 260, the fluid 260 may percolate the contiguous free space 220 of the dosage form 200 and the open pores 245 in the fibers 210, FIGS. 2b and 2c. Subsequently, the dissolution fluid 260 may diffuse into the excipient 240 and plasticize it, forming a viscous suspension 250. Because the excipient 240 weight fraction in the fibers 210 can be very small, the concentration of excipient molecules 241 in the suspension 250 may also be small.

Thus, if the excipient 240, 241 molecular weight is small, and the disentanglement concentration of the excipient 240, 241 very large, the excipient molecules 241 in the suspension 250 may be disentangled, and the suspension 250 may be so fluidic that it rapidly deforms, fragments, and dissolves, FIG. 2b. By contrast, if the excipient 240, 241 molecular weight is very large, and the disentanglement concentration of the excipient 240, 241 very small, the excipient molecules 241 in the suspension 250 may be entangled, and a highly viscous mass 252 may be formed that erodes and releases drug 230 slowly, FIG. 2b.

Therefore, by appropriate choice of the quantity and properties of the water-soluble polymeric excipient 240, control of the drug 230 release rate by dosage forms 200 with close-packed fibers 210 and high drug 230 loading may be feasible within a large range. The pores 245 in the fibers 210 enable to reduce the required quantity of water-soluble polymeric excipient 240, and hence increase the range of the drug release rate of the dosage form.

### SUMMARY OF THE INVENTION

Generally, the dosage forms disclosed herein comprise a three-dimensional structural framework of repeatably arranged, close-packed structural elements. The elements include active ingredient particles, an excipient matrix to bind and carry said particles, and pores.

More specifically, in one aspect the invention herein comprises a pharmaceutical dosage form comprising a drug-containing solid having an outer surface and an internal structure contiguous with and terminating at said outer surface; said internal structure comprising a three dimensional structural framework one or more repeatably arranged, fibrous elements; said fibrous elements occupying a volume fraction of the drug-containing solid in the range of 0.45 to 0.98; and said fibrous elements comprising an excipient matrix with drug particles and pores dispersed through their volume; wherein the volume fraction of pores in at least one fibrous element is in the range of 0.01 to 0.4 (e.g., in the range of 0.02-0.3).

In some embodiments, the three dimensional structural framework comprises a plurality of criss-crossed stacked layers of fibers.

In some embodiments, said one or more fibers further comprise fiber segments spaced apart from adjoining segments by free spacings defining one or more free spaces through the drug-containing solid.

In some embodiments, said excipient matrix comprises at least a polymer that is soluble in a physiological fluid.

In some embodiments, said excipient matrix including at least one physiological fluid-soluble polymer, wherein the mass of physiological fluid-soluble polymeric excipient in the drug-containing solid divided by the volume of free space in the drug-containing solid is no greater than six times (e.g., no greater than five times, or no greater than four times) the disentanglement concentration of said physiological fluid-soluble polymeric excipient in said physiological fluid.

In some embodiments, the volume fraction or weight fraction of said dispersed drug particles in at least one fiber or fiber segment is greater than 0.5 (e.g., greater than 0.55, or greater than 0.6).

In some embodiments, at least a pore is filled with gas.

In another aspect, the invention herein comprises a pharmaceutical dosage form comprising a drug-containing solid having an outer surface and an internal structure contiguous with and terminating at said outer surface said internal structure comprising a plurality of criss-crossed stacked layers of fibers; said fibers comprising fiber segments spaced apart from adjoining fiber segments by free spacings defining one or more free spaces through the drug-containing solid, wherein the volume fraction of fibers in the drug-containing solid is in the range between 0.45 and 0.98; said fibers further comprising an excipient matrix with drug particles and pores dispersed throughout the fiber volume; said excipient matrix including at least one physiological fluid-soluble polymer, wherein the mass of physiological fluid-soluble polymeric excipient in the drug-containing solid divided by the volume of free space in the drug-containing solid is no greater than six times the disentanglement concentration of said physiological fluid-soluble polymeric excipient in said physiological fluid; whereby the volume fraction of said dispersed drug particles in at least one fiber or fiber segment is greater than 0.5; said pores are filled with at least a gas; and the volume fraction of pores in at least one fiber is in the range between 0.01 and 0.4.

In some embodiments, one or more free spaces are interconnected through the thickness of the drug-containing solid.

In some embodiments, one or more interconnected free spaces extend over a length at least equal to the thickness of the drug-containing solid.

In some embodiments, the free space is contiguous.

In some embodiments, the effective free spacing between segments across one or more interconnected free spaces on average is in the range of 5 μm-1.5 mm (e.g., 10 μm-1.5 mm, or 10 μm-1 mm).

In some embodiments, the free spacing between segments of the one or more fibers is precisely controlled.

In some embodiments, upon immersion in a physiological fluid, said fluid percolates at least an interconnected free space, and the structural framework transitions to viscous and fragments, promoting dissolution of the active ingredient.

In some embodiments, one or more fibers comprise a continuous solid matrix through their thickness.

In some embodiments, average thickness of the one or more fibers is in the range of 5 μm to 2.5 mm (e.g., 10 μm to 2 mm, or 15 μm to 1.75 mm).

In some embodiments, the fibers occupy a volume fraction of the drug-containing solid in the range of 0.6 to 0.98.

In some embodiments, the volume fraction or weight fraction of dispersed drug particles in at least one fiber or fiber segment is greater than 0.6.

In some embodiments, the volume fraction or weight fraction of dispersed drug particles in a three dimensional framework of fibers is greater than 0.6.

In some embodiments, at least one physiological fluid-soluble excipient is absorptive of a physiological/body fluid, and wherein rate of penetration of the physiological/body fluid into a fiber or said absorptive excipient under physiological conditions is greater than the average fiber thickness divided by 3600 seconds.

In some embodiments, the least one physiological fluid-soluble excipient comprises a solubility greater than 5 g/l in an aqueous physiological/body fluid under physiological conditions.

In some embodiments, at least one physiological fluid-soluble excipient is selected from the group comprising hydroxypropyl methylcellulose, hydroxyethyl cellulose, polyvinyl alcohol, polyvinylpyrrolidone, hydroxypropyl methylcellulose acetate succinate, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), or vinylpyrrolidone-vinyl acetate copolymer.

In some embodiments, the molecular weight of at least one physiological fluid-soluble excipient is in the range of 1 kg/mol to 200 kg/mol (e.g., 1 kg/mol-100 kg/mol, or 2 kg/mol-100 kg/mol).

In some embodiments, at least one physiological fluid-soluble excipient comprises hydroxypropyl methylcellulose.

In some embodiments, the molecular weight of said hydroxypropyl methylcellulose excipient is in the range between 1 kg/mol and 80 kg/mol (e.g., between 2 kg/mol and 80 kg/mol, or between 2 kg/mol and 50 kg/mol).

In some embodiments, the volume or weight fraction of physiological fluid-soluble polymer in a fiber is in the range between 0.02 and 0.35.

In some embodiments, one or more fibers comprise at least a pore with a closed end.

In some embodiments, upon immersion of one or more elements in a dissolution fluid a capillary pressure develops in said at least one pore with a closed end.

In some embodiments, the pores in the fibers comprise a volume fraction in the range of 0.04 to 0.3 (e.g., 0.05 to 0.3, or 0.05 to 0.25).

In some embodiments, the pores in the fibers comprise an average size (e.g., an average width or average diameter) in the range of 0.5 μm to 125 μm.

In some embodiments, at least one fiber comprises a surface-connected pore.

In some embodiments, the pores in the fibers are randomly or almost randomly distributed.

In some embodiments, at least one pore is filled with a matter comprising air.

In another aspect, the invention herein comprises a pharmaceutical dosage form comprising a drug-containing solid having an outer surface and an internal structure contiguous with and terminating at said outer surface; said internal structure comprising a plurality of criss-crossed stacked layers of fibers; said fibers comprising fiber segments spaced apart from adjoining fiber segments by free spacings defining one or more free spaces through the drug-containing solid, wherein the volume fraction of fibers in the drug-containing solid is in the range between 0.55 and 0.98; said fibers further comprising an excipient matrix with drug particles and pores dispersed throughout the fiber volume; said excipient matrix including at least one physiological fluid-soluble polymer, wherein the mass of physiological fluid-soluble polymeric excipient in the drug-containing solid divided by the volume of free space in the drug-containing solid is no greater than six times the disentanglement concentration of said physiological fluid-soluble polymeric excipient in said physiological fluid; whereby the volume or weight fraction of said dispersed drug particles in at least one fiber or fiber segment is greater than 0.6; the volume or weight fraction of said physiological fluid-soluble polymeric excipient in at least one fiber or fiber segment is in the range of 0.04 to 0.4; said pores are filled with at least a gas; and the volume fraction of pores in at least one fiber is in the range between 0.01 and 0.4.

Embodiments or parts of embodiments described with respect to one aspect of the invention can be applied with respect to another aspect. By way of example but not by way of limitation, certain embodiments of the claims described with respect to the first aspect can include features of the claims described with respect to the second or third aspect, and vice versa.

This invention may be better understood by reference to the accompanying drawings, attention being called to the fact that the drawings are primarily for illustration, and should not be regarded as limiting.

### BRIEF DESCRIPTION OF THE DRAWINGS

The objects, embodiments, features, and advantages of the present invention are more fully understood when considered in conjunction with the following accompanying drawings:

FIG. 1 schematically illustrates a non-limiting manufacturing process of dosage forms as disclosed herein: (a) material preparation and 3D-micro-patterning, and (b) evolution of the microstructure in a fiber during drying;

FIG. 2 presents non-limiting examples of a pharmaceutical dosage forms according to the invention herein, and their disintegration upon immersion in a physiological or body fluid under physiological conditions;

FIG. 3 illustrates a non-limiting schematic of a pharmaceutical dosage form according to the invention herein;

FIG. 4 presents non-limiting schematics of the disintegration mechanism of dosage forms as disclosed herein after immersion in a physiological or body fluid under physiological conditions;

FIG. 5 is a non-limiting schematic illustrating pressure in a pore and stresses in a pore wall: (a) longitudinal section of an idealized cylindrical pore with one end open and the other closed and (b) cross section;

FIG. 6 presents non-limiting schematics of fiber and dosage form disintegration: (a) fibrous dosage form with excipient of small molecular weight, and (b) fibrous dosage form with excipient of large molecular weight;

FIG. 7 presents a non-limiting dosage form according to the invention herein along with its microstructure;

FIG. 8 presents a non-limiting fibrous microstructure of a dosage form herein, and a histogram of the length of fiber segments between adjacent contacts;

FIG. 9 shows another non-limiting fibrous microstructure herein, and a histogram of the angle between contacting fibers;

FIG. 10 schematically illustrates a non-limiting microstructure of an element (e.g., a fiber) as as disclosed herein;

FIG. 11 is a non-limiting schematic of a solvent-based method of manufacturing dosage forms according to this invention;

FIG. 12 presents scanning electron micrographs of the microstructures of non-limiting experimental fibrous dosage forms: (a) top view and (b) front view of dosage form A, HPMC 10k excipient, (c) top view and (d) front view of dosage form B, HPMC 26k excipient, and (e) top view and (f) front view of dosage form C, HPMC 86k excipient;

FIG. 13 presents scanning electron micrographs of the cross section of fibers in non-limiting experimental fibrous dosage forms: (a) dosage form A, HPMC 10k excipient, and (b) dosage form C, HPMC 86k excipient;

FIG. 14 depicts images of non-limiting experimental dosage forms after immersion in a dissolution fluid: (a) dosage form A, HPMC 10k excipient, (b) dosage form B, HPMC 26k excipient, and (c) dosage form C, HPMC 86k excipient.

FIG. 15 presents results of drug release by non-limiting experimental fibrous dosage forms: (a) fraction of drug released versus time, and (b) Log-log plot of the fraction of drug released. The fit equations of the log-log plots are: (A) m_{d}/M₀=0.084t^{0.94}, R²=0.98; (B) m_{d}/M₀=0.0157t^{0.95}, R²=0.98, and (C) m_{d}/M₀=0.0047t^{0.87}, R²=0.97;

FIG. 16 shows a log-log plot of the time to release 80% of the amount of drug in the dosage form, t_{0.8}, versus molecular weight of the excipient, M_{w};

FIG. 17 presents shear viscosity of HPMC-water solutions, μₛₒₗ, at various concentrations of the excipient, cₑ, at a shear rate of l/s. The fit equations are listed in the experimental example 5 titled “Shear viscosity of excipient-water solutions”;

FIG. 18 plots disentanglement concentration, cₑ*, of the non-limiting excipient used in the experimental dosage forms versus molecular weight, M_{w};

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “one or more active ingredients” and “drug” are used interchangeably. As used herein, an “active ingredient” or “active agent” or “drug” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

In the invention herein, a drug-containing solid generally comprises a solid that includes or contains at least a drug. A drug-containing solid generally can have any shape, geometry, or form.

Furthermore, in the context of some embodiments herein, a three dimensional structural framework (or network) of one or more elements comprises a structure (e.g., an assembly or an assemblage or an arrangement or a skeleton or a skeletal structure or a three-dimensional lattice structure of one or more drug-containing elements) that extends over a length, width, and thickness greater than 100 μm. This includes, but is not limited to structures that extend over a length, width, and thickness greater than 200 μm, or greater than 300 μm, or greater than 500 μm, or greater than 700 μm, or greater than 1 mm, or greater than 1.25 mm, or greater than 1.5 mm, or greater than 2 mm.

In other embodiments, a three dimensional structural framework (or network) of elements may comprise a structure (e.g., an assembly or an assemblage or a skeleton or a skeletal structure of one or more elements) that extends over a length, width, and thickness greater than the average thickness of at least one element (or at least one segment) in the three dimensional structural framework (or network) of elements. This includes, but is not limited to structures that extend over a length, width, and thickness greater than 1.5, or greater than 2, or greater than 2.5, or greater than 3, or greater than 3.5, or greater than 4 times the average thickness of at least one element (or at least one segment) in the three dimensional structural framework (or network) of elements.

In some embodiments, a three dimensional structural framework (or network) of elements is continuous. Furthermore, in some embodiments, one or more elements or segments thereof are bonded to each other or interpenetrating.

It may be noted that the terms “three dimensional structural network”, “three dimensional structural framework”, and “three dimensional lattice structure” are used interchangeably herein. Also, the terms “three dimensional structural framework of drug-containing elements”, “three dimensional structural framework of elements”, “three dimensional structural framework of one or more elements”, “three dimensional framework of elements”, “three dimensional structural framework of fibers”, “three dimensional framework”, “structural framework”, etc. are used interchangeably herein.

In the invention herein, a “structural element” or “element” refers to a two-dimensional element (or 2-dimensional structural element), or a one-dimensional element (or 1-dimensional structural element), or a zero-dimensional element (or 0-dimensional structural element).

As used herein, a two-dimensional structural element is referred to as having a length and width much greater than its thickness. In the present disclosure, the length and width of a two-dimensional sructural element are greater than 2 times its thickness. An example of such an element is a “sheet”. A one-dimensional structural element is referred to as having a length much greater than its width and thickness. In the present disclosure, the length of a one-dimensional structural element is greater than 2 times its width and thickness. An example of such an element is a “fiber”. A zero-dimensional structural element is referred to as having a length and width of the order of its thickness. In the present disclosure, the length and width of a zero-dimensional structural element are no greater than 2 times its thickness. Furthermore, the thickness of a zero-dimensional element is less than 2.5 mm. Examples of such zero-dimensional elements are “particles” or “beads” and include polyhedra, spheroids, ellipsoids, or clusters thereof.

Moreover, in the invention herein, a segment of a one-dimensional element is a fraction of said element along its length. A segment of a two-dimensional element is a fraction of said element along its length and/or width. A segment of a zero-dimensional element is a fraction of said element along its length and/or width and/or thickness. The terms “segment of a one-dimensional element”, “fiber segment”, “segment of a fiber”, and “segment” are used interchangeably herein. Also, the terms “segment of a two-dimensional element” and “segment” are used interchangeably herein. Also, the terms “segment of a zero-dimensional element” and “segment” are used interchangeably herein.

As used herein, the terms “fiber”, “fibers”, and “one or more fibers”, are used interchangeably. They are understood as the solid, structural elements (or building blocks) that make up part of or the entire three dimensional structural framework or network (e.g., part of or the entire dosage form structure, or part of or the entire structure of a drug-containing solid, etc.). A fiber has a length much greater than its width and thickness. In the present disclosure, a fiber is referred to as having a length greater than 2 times its width and thickness (e.g., the length is greater than 2 times the fiber width and the length is greater than 2 times the fiber thickness). This includes, but is not limited to a fiber length greater than 3 times, or greater than 4 times, or greater than 5 times, or greater than 6 times, or greater than 8 times, or greater than 10 times, or greater than 12 times the fiber width and thickness. In other embodiments that are included but not limiting in the disclosure herein, the length of a fiber may be greater than 0.3 mm, or greater than 0.5 mm, or greater than 1 mm, or greater than 2.5 mm.

Moreover, as used herein, the term “fiber segment” or “segment” refers to a fraction of a fiber along the length of said fiber.

In the invention herein, fibers (or fiber segments) may be bonded, and thus they may serve as building blocks of “assembled structural elements” with a geometry different from that of the original fibers. Such assembled structural elements include two-dimensional elements, one-dimensional elements, or zero-dimensional elements.

In the invention herein, drug release from a solid element (or a solid dosage form, or a solid matrix, or a drug-containing solid) refers to the conversion of drug (e.g., one or more drug particles, or drug molecules, or clusters thereof, etc.) that is/are embedded in or attached to the solid element (or the solid dosage form, or the solid matrix, or three dimensional structural framework, or the drug-containing solid) to drug in a dissolution medium.

As used herein, the terms “dissolution medium”, “physiological fluid”, “body fluid”, “dissolution fluid”, “medium”, “fluid”, “aqueous solution”, and “penetrant” are used interchangeably. They are understood as any fluid produced by or contained in a human body under physiological conditions, or any fluid that resembles a fluid produced by or contained in a human body under physiological conditions. Generally, a dissolution fluid contains water and thus may be aqueous. Examples include, but are not limited to: water, saliva, stomach fluid, gastrointestinal fluid, saline, etc. at a temperature of 37° C. and a pH value adjusted to the relevant physiological condition.

In the invention herein, moreover, a “relevant physiological fluid” is understood as the relevant physiological fluid surrounding the dosage form in the relevant physiological application. For example, if the dosage form is a gastroretentive dosage form, a relevant physiological fluid is gastric fluid.

Further information related to the definition, characteristics, features, composition, analysis etc. of the disclosed dosage forms, and the elements for fabricating or constructing them, is provided throughout this specification.

### Scope of the Invention

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

By way of example but not by way of limitation, it is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Furthermore, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting

### DETAILED DESCRIPTION OF THE INVENTION

### Aspects of the Dosage Form

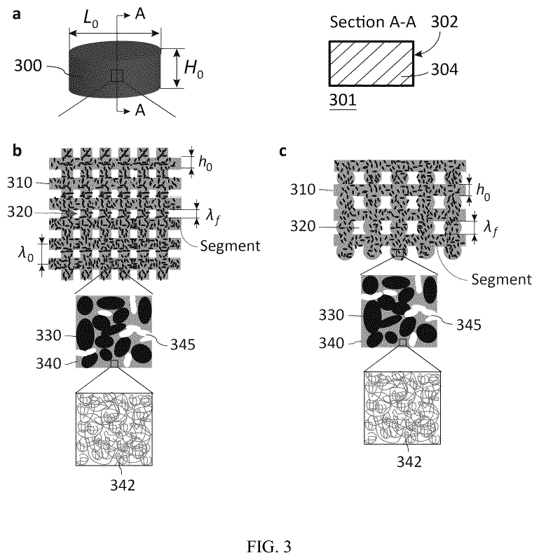

FIGS. 3a-3c present a non-limiting example of a pharmaceutical dosage form 300 according to the invention herein. The dosage form 300 comprises a drug-containing solid 301 having an outer surface 302 and an internal structure 304. The internal structure 304 comprises a three dimensional structural framework of one or more repeatably arranged, extruded structural elements 310. The extruded structural elements 310 occupy a volume fraction in the drug-containing solid 301 in the range of 0.45 to 0.98. The extruded structural elements 310 further comprise an excipient matrix 340 with drug particles 330 and pores 345 dispersed through their volume. The volume fraction of pores 345 in at least one element 310 may be in the range between 0.01 and 0.4.

Generally, structural elements 310 are understood as “repeatably arranged” if such structural features as spacing between elements 310, orientation of elements 310, etc. are controlled. A structural feature is referred to as “controlled” if a standard deviation of said feature across a three dimensional structural framework (or across multiple three dimensional structural frameworks of multiple dosage forms, etc.) is smaller (or much smaller) than that in a random structure with randomly arranged elements 310.

An element 310 is understood as “extruded” if it is produced by extrusion, which is generally referred to as “produced by forcing material through a die”. A typical feature of an extruded element 310 includes a fairly constant cross section along its length. A non-limiting example of an extruded structural element is a fiber.

In the invention herein, the “volume fraction of extruded structural elements in a drug-containing solid” is referred to as the volume of said extruded structural elements in said drug-containing solid divided by the volume of said drug-containing solid.

In the invention herein, moreover, “drug particles” are generally referred to as particles including at least a drug or active ingredient.

In preferred embodiments, extruded structural elements may further comprise segments spaced apart from adjoining segments by free spacings defining one or more free spaces through the drug-containing solid.

Also, in preferred embodiments said excipient matrix includes at least one physiological fluid-soluble polymer, wherein the mass of the physiological fluid-soluble polymeric excipient in the drug-containing solid divided by the volume of free space in the drug-containing solid is no greater than six times the disentanglement concentration of said physiological fluid-soluble polymeric excipient in said physiological fluid.

In preferred embodiments, moreover, the volume fraction of said dispersed drug particles in at least one element or segment is greater than 0.5. Also, in preferred embodiments, said pores are filled with at least a gas.

FIGS. 3a-3c also illustrate another non-limiting example of a pharmaceutical dosage form 300 according to the invention herein. The dosage form 300 comprises a drug-containing solid 301 having an outer surface 302 and an internal structure 304. The internal structure 304 comprises a three dimensional structural framework of one or more repeatably arranged fibers 310. The fibers 310 occupy a volume fraction in the drug-containing solid 301 in the range of 0.45 to 0.98. The fibers 310 further comprise an excipient matrix 340 with drug particles 330 and pores 345 dispersed through their volume. The volume fraction of pores 345 in at least one fiber 310 may be in the range between 0.01 and 0.4.

In preferred embodiments, said internal structure comprises a plurality of criss-crossed stacked layers of fibers. Said fibers may further comprise fiber segments spaced apart from adjoining fiber segments by free spacings defining one or more free spaces through the drug-containing solid.

In preferred embodiment, moreover, said excipient matrix includes at least one physiological fluid-soluble polymer, wherein the mass of the physiological fluid-soluble polymeric excipient in the drug-containing solid divided by the volume of free space in the drug-containing solid is no greater than six times the disentanglement concentration of said physiological fluid-soluble polymeric excipient in said physiological fluid.

Furthermore, in preferred embodiments the volume fraction of said dispersed drug particles in at least one fiber or fiber segment is greater than 0.5. In preferred embodiments, moreover, said pores are filled with at least a gas.

FIGS. 3a-3c illustrate a further non-limiting example of a pharmaceutical dosage form 300 according to the invention herein. The dosage form 300 comprises a drug-containing solid 301 having an outer surface 302 and an internal structure 304. The internal structure 304 comprises a plurality of criss-crossed stacked layers of fibers 310. Said fibers 310 comprise an excipient matrix 340 with drug particles 330 and pores 345 dispersed throughout the fiber 310 volume. Said fibers 310 further comprise fiber segments spaced apart from adjoining fiber segments by free spacings, f, defining one or more free spaces 320 through the drug-containing solid 301, wherein the volume fraction of fibers 310 in the drug-containing solid 301 is in the range between 0.45 and 0.98. Said excipient matrix 340 includes at least one physiological fluid-soluble polymer 342, wherein the mass of the physiological fluid-soluble polymeric excipient 342 in the drug-containing solid 301 divided by the volume of free space 320 in the drug-containing solid 301 is no greater than six times the disentanglement concentration of said physiological fluid-soluble polymeric excipient 342 in said physiological fluid. The volume fraction of said dispersed drug particles 330 in at least one fiber 310 or fiber segment is greater than 0.5. Also, said pores 345 are filled with at least a gas, and the volume fraction of pores 345 in at least one fiber 310 is in the range between 0.01 and 0.4.

It may be noted that in preferred embodiments, average thickness of one or more elements or one or more fibers generally is in the range between about 5 μm and 2.5 mm.

Additional non-limiting examples of three dimensional structural frameworks with interconnected free spaces illustrating how the fibers may be structured, arranged, or assembled are, for example, disclosed in the co-pending U.S. application Ser. No. 15/482,776 titled “Fibrous dosage form”. More examples of how fibers or elements may be structured or arranged in the three dimensional structural network or framework of one or more fibers or elements would be obvious to a person of ordinary skill in the art. All of them are within the spirit and scope of this invention.

### Models for Dosage Form Design

The following examples present non-limiting ways by which the drug release and disintegration behavior of the disclosed dosage forms may be modeled. They will enable one of skill in the art to more readily understand the details and advantages of the invention. The models and examples are for illustrative purposes only, and are not meant to be limiting in any way.

### (a) Microstructures and Formulations

As shown schematically in the non-limiting FIGS. 3a-3c, the non-limiting dosage forms 300 modeled herein comprise a drug-containing solid 301 having an outer surface 302 and an internal structure 304. The internal structure 304 comprises a plurality of criss-crossed stacked layers of fibers 310 with average thickness, h₀, in the range between 5 μm and 2.5 mm. Said fibers 310 comprise fiber segments spaced apart from adjoining fiber segments by free spacings, λ_{f}, defining one or more free spaces 320 through the drug-containing solid 301. The volume fraction of fibers 310 in the drug-containing solid 301 is in the range between 0.55 and 0.98. Said fibers 310 further comprise an excipient matrix 340 with drug particles 330 and pores 345 dispersed through the fiber 310 volume. Said excipient matrix 340 includes at least one physiological fluid-soluble polymer 342, wherein the mass of the physiological fluid-soluble polymeric excipient 342 in the drug-containing solid 301 divided by the volume of free space 320 in the drug-containing solid 301 is no greater than six times the disentanglement concentration of said physiological fluid-soluble polymeric excipient 342 in said physiological fluid. The volume fraction of said dispersed drug particles 330 in at least one fiber 310 or fiber segment is greater than 0.6. Also, said pores 345 are filled with at least a gas, and the volume fraction of pores 345 in at least one fiber 310 is in the range between 0.01 and 0.4.

In the specific non-limiting dosage forms modeled herein, moreover, the physiological fluid-soluble polymeric excipient 342 comprises hydroxypropyl methylcellulose (HPMC). Three different dosage forms with three different, non-limiting excipient molecular weights are considered: dosage form A comprising HPMC of molecular weight 10 kg/mol (HPMC 10k), dosage form B comprising HPMC of molecular weight 26 kg/mol (HPMC 26k), and dosage form C comprising HPMC of molecular weight 86 kg/mol (HPMC 86k).

### (b) Overview of Drug Release Mechanisms

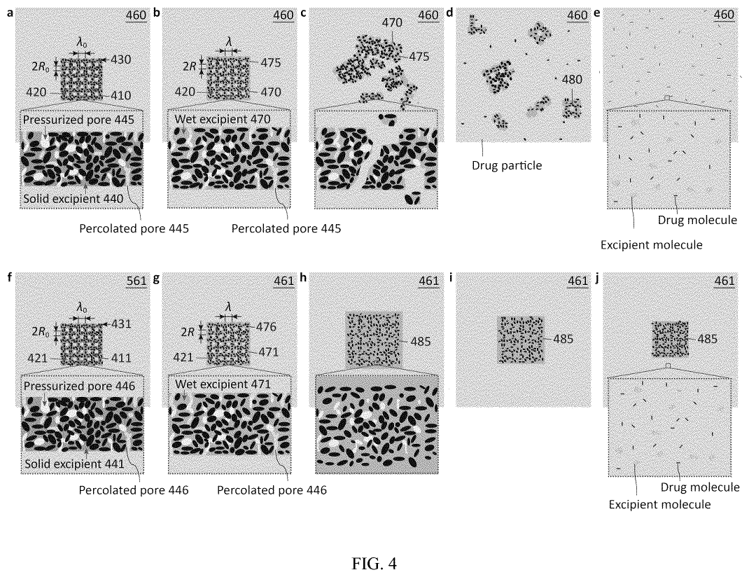

Upon immersion of the dosage form with HPMC 10k excipient (e.g., the first dosage form or “dosage form A”) in a dissolution fluid 460, the fluid 460 may percolate free space 420 because the excipient 440 in the fibers 410 is fairly hydrophilic and the free space 420 is contiguous, FIG. 4a. The fluid 460 may then percolate surface-connected pores 445 in the fibers 410 with two open ends, and develop a capillary pressure, p_{cap}, in pores 445 with one closed end, as shown in the inset of FIG. 4a. Also, the fluid 460 and the excipient 440 may inter-diffuse, and the fibers 410 may transition from solid to a fluidic or viscous suspension 475 comprising drug particles 430 embedded in a viscous excipient-dissolution fluid solution 470, FIG. 4b. The viscosity of the fluidized fibers 475 may be fairly low because the molecular weight of the excipient 440 is small. Thus, due to the internal capillary pressure and due to other forces the low-viscosity, fluidized fibers 475 may deform and fragment, FIG. 4c. The thin fragments 480 may then erode rapidly into the dissolution fluid 460, and release drug molecules and drug particles 430, which then dissolve, FIGS. 4d-4e.

The disintegration mechanism of the dosage form with HPMC 26k excipient (e.g., the second dosage form or “dosage form B”) may be similar to that of dosage form A. But because the molecular weight of the excipient is greater, the rates of fragmentation and erosion may be smaller.

Upon immersion of the dosage form with HPMC 86k excipient (e.g., the third dosage form or “dosage form C”), too, the dissolution fluid 461 may percolate free spaces 421 between fibers 511 and the surface-connected, open pores 446 of the fibers 411, and a capillary pressure may develop in pores 446 with a closed end, FIG. 4f. Also, dissolution fluid (e.g., water) 461 and excipient 441 may inter-diffuse, and the fibers 411 may transition from solid 411 to a viscous dispersion 476 comprising drug particles 431 and a viscous excipient-dissolution fluid solution 471, FIG. 4g. However, because the molecular weight of the excipient 441 is very large, the viscosity of the excipient-dissolution fluid solution 471 and the fluid-penetrated fibers 476 may be far greater than in the previous cases. Consequently, the capillary pressure in the fibers 411, 476 (e.g., the pressure in the pores of the fibers) may not break up the structure 476, and a monolithic, thick viscous mass 485 may be formed, FIG. 4h. The thick viscous mass 485 may slowly erode into the dissolution fluid 461 from the surface, FIGS. 4i and 4j, thereby releasing drug molecules and drug particles, which eventually may dissolve.

The sequence of steps that may lead to drug release are modeled below for all three dosage forms.

(c) Percolation of the Dissolution Fluid into the Dosage Form and the Open Pores in the Fibers

Without wishing to be bound to a particular theory, it is believed that a gas-filled free space, channel, network of channels, etc. is percolated rapidly by a dissolution fluid if (a) at least one (and preferably at least two) open ends of said gas-filled free space, channel, network of channels, etc. are in contact (e.g., in direct contact) with said dissolution fluid, (b) the surface of said gas-filled free space, channel, network of channels, etc. is hydrophilic or highly hydrophilic, and (c) the channel width or height (e.g., the channel diameter, or the width of the free space, or the width or diameter of the pores, etc.) is at the micro- or macro-scale. In the invention herein, a channel width or height or diameter is understood as “at the micro- or macro-scale) if it is greater than about 1 μm. This includes but is not limited to a channel width or height or diameter greater than 2 μm, or greater than 5 μm, or greater than 7 μm, or greater than 10 μm, or greater than 15 μm, or greater than 20 μm.

A rough estimate of the percolation time may be obtained if the free space of the dosage form and the open pores in the fibers are treated as a collection of capillary conduits exposed to the dissolution medium at one end and to air at the other. The percolation time, t_{perc}, may then be expressed by the Lucas-Washburn equation:

$${t}_{p e r c} = \frac{2 {l}_{p e r c}^{2} {μ}_{w}}{γ {r}_{c o n d} cos {θ}_{c}} \qquad ( 1 )$$

where l_{perc} is the percolation length, r_{cond} the radius of a conduit, γ the surface tension of the dissolution fluid, and θ_{c} the contact angle.

If the radius of the conduits in the free space between fibers is about 85 μm, and the non-limiting parameters l_{perc}˜ 3 mm, μ_{w}˜ 0.001 Pa·s, γ˜0.072 N/m, and θ_{c}˜30°, by Eq. (1) the percolation time of the free space between fibers is about 3.4 ms.

Similarly, if the diameter of the pores in the fibers is about 5-50 μm, and the percolation length of a surface-connected pore is about 180 μm (about equal to the fiber radius), the calculated percolation time of a surface-connected, open pore in a fiber, by Eq. (1), is less than about 1 ms.

Thus, with appropriate microstructure and contact angle, percolation into a free space of the dosage form or into a pore of a fiber may generally be very fast.

More models of fluid percolation into a free space of the dosage form or into a pore of a fiber would be obvious to a person of ordinary skill in the art. All of them are within the spirit and scope of this disclosure.

### (d) Capillary Pressure in the Pores of the Fibers

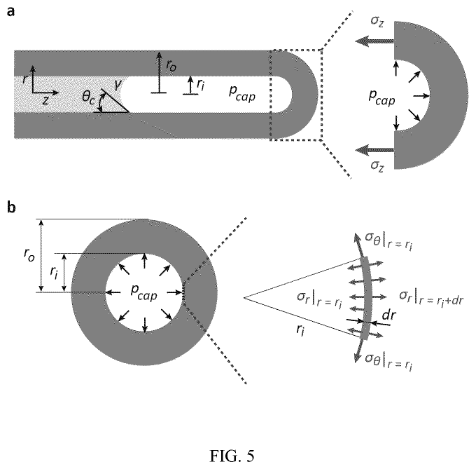

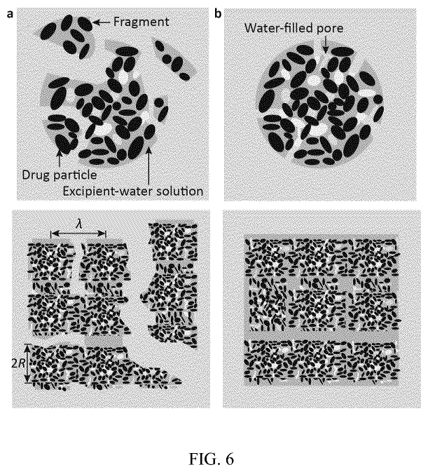

In contrast to the open pores in the fibers, the air or gas in pores with one end open and one end closed may be compressed, FIGS. 5a and 6a. The capillary pressure, p_{cap}, in the pores may be written as:

$${p}_{cap} = \frac{2 γ cos {θ}_{c}}{{r}_{i}} \qquad ( 2 )$$

where γ is the surface tension of the dissolution fluid, θ_{c} the contact angle, and rᵢ the inner radius of the pore.

The air pressure induces a stress in the wall of the pore. An in-depth derivation of the stress field is far beyond the scope of this disclosure, but if the pores and pore walls are assumed cylindrical vessels, as shown in FIG. 5a, reasonable engineering estimates of the stresses may be obtained.

By force balance the principal stress in axial direction, σ_{z}, in the cylinder wall (or “pore wall” or “wall”) may be written as:

$${σ}_{z} = \frac{{r}_{i}^{2}}{{r}_{o}^{2} - {r}_{i}^{2}} {p}_{cap} {r}_{i} \leq r \leq {r}_{o} \qquad ( 3 a )$$

where rₒ is the outer radius of the wall.

Similarly, from Lame's equations the principal stresses in radial and tangential directions, σᵣ and σ_{θ} (shown in FIG. 5b), may be greatest on the internal surface of the cylinder wall (or “pore wall” or “wall”). They may be written as:

$${{σ}_{r} \vert}_{r = {r}_{i}} = - {p}_{cap} \qquad ( 3 b )$$

$${{σ}_{θ} \vert}_{r = {r}_{i}} = \frac{{r}_{i}^{2} + {r}_{o}^{2}}{{r}_{o}^{2} - {r}_{i}^{2}} {p}_{cap} \qquad ( 3 c )$$

Substituting Eq. (3) in Eqs. (4) the principal stresses in the wall may be expressed as:

$${{σ}_{r} \vert}_{r = {r}_{i}} = - \frac{2 γ cos {θ}_{c}}{{r}_{i}} \qquad ( 4 a )$$

$${{σ}_{r} \vert}_{r = {r}_{i}} = \frac{{r}_{i}^{2} + {r}_{o}^{2}}{{r}_{o}^{2} - {r}_{i}^{2}} \frac{2 γcos {θ}_{c}}{{r}_{i}} \qquad ( 4 b )$$

$${σ}_{z} = \frac{{r}_{i}^{2}}{{r}_{o}^{2} - {r}_{i}^{2}} \frac{2 γcos {θ}_{c}}{{r}_{i}} {r}_{i} \leq r \leq {r}_{o} \qquad ( 4 c )$$

The outer and inner radii of the cylinder may further be related to the pore volume fraction, φₚₒᵣₑ, by:

$${φ}_{pore} = \frac{{r}_{i}^{2}}{{r}_{o}^{2}} \qquad ( 5 )$$

Using the non-limiting parameters rᵢ˜2.5-25 μm, γ˜0.072 N/m, φₚₒᵣₑ˜0.12, and θ_{c}˜30°, by Eqs. (4) and (5) the principal stresses σᵣ, σ_{θ}, and σ_{z} are about −50, 63, and 6.8 kPa, respectively.

The yield and tensile strengths of pharmaceutical solids generally are higher than 1 MPa—much greater than the stresses calculated above. Thus, the stresses due to the capillary pressure may be too small to fragment the solid fibers. However, as shown later the fibers may fragment after interdiffusion with water and transitioning to viscous.

For further information related to the mechanical properties (e.g., fracture strength, yield strength, elastic modulus, etc.) of pharmaceutical solids, see, e.g., J. T. Fell, J. M. Newton, J. Pharm. Sci. 59 (1973) 688-691; C. K. Tye, C. Sun, G. E. Amidon, J. Pharm. Sci., 94 (2005) 465-472; or A. H. Blaesi, N. Saka, Int. J. Pharm. 509 (2016) 444-453. More models for estimating the stresses in the pore walls of the fibers obvious to a person of ordinary skill in the art are all within the spirit and scope of this disclosure.

(e) Diffusion of Dissolution Fluid into the Fibers

After partial or full percolation of the free space of the dosage form, the fluid surrounding the fibers may diffuse into the excipient matrix in the fiber. As the water or fluid concentration increases in the fiber increases the excipient may swell and transition to viscous. An in-depth analysis of the diffusion problem through a porous, composite fiber is well beyond the scope of this disclosure. But if the fiber is assumed pore-free and homogeneous, and the water diffusivity, D_{w}, and the boundary concentration, c_{b}, are constant, the water concentration, c_{w}, in a fiber as a function of time, t, and radial position, r, may be expressed as:

$$\frac{{c}_{w}}{{c}_{b}} = 1 - \frac{2}{{R}_{0}} {\sum }_{n = 1}^{∞} \frac{exp ( - {D}_{w} {α}_{n}^{2} t ) {J}_{0} ( r {α}_{n} )}{{α}_{n} {J}_{1} ( {R}_{0} {α}_{n} )} \qquad ( 8 )$$

where R₀ is the initial fiber radius (e.g., the radius of the solid fibers), and J₀ and J₁ are the Bessel functions of the first kind of order zero and one, respectively, and the αₙ's are the roots of

J₀(R₀αₙ)=0 (9)

A graphical solution to Eqs. (8) and (9) presented by Crank (see, e.g., J. Crank, “The Mathematics of Diffusion”, second edition, Oxford University Press, 1975) suggests that the concentration at the center of a fiber may reach about 66 percent of the boundary concentration if R₀²/D_{w}t≈0.3. Thus, a rough estimate of the time the water (or dissolution fluid) molecules require to penetrate to the center of a fiber may be written as:

$${t}_{pen} ≅ 0.3 \frac{{R}_{0}^{2}}{{D}_{w}} \qquad ( 10 )$$

For the non-limiting parameter values R₀˜180 μm and D_{w}˜3×10⁻¹¹ m²/s, tₚₑₙ˜ 6 min. Thus, if the fibers are thin and the diffusivity of water in the fibers is fairly large, the penetration time is fairly small. The penetration time may be even smaller if the pores in the fiber are highly connected.

### (f) Formation of a Viscous Suspension

The fibers may expand as water diffuses in, and they may transition to a viscous suspension consisting of drug particles, air-filled pores, and a viscous excipient-water solution. If fiber expansion is predominantly in radial direction, the fibers coalesce if the radius, R≈λ₀/2. Moreover, if the densities of the excipient, drug, and dissolution fluid are the same, and the volume fraction of pores is small, after fiber coalescence the weight fractions of drug, excipient, and dissolution fluid (water) in the viscous suspension may be estimated by:

$${w}_{d , v} = {{w}_{d , s} ( \frac{2 {R}_{0}}{{λ}_{0}} )}^{2} \qquad ( 11 a )$$

$${w}_{e , v} = {{w}_{e , s} ( \frac{2 {R}_{0}}{{λ}_{0}} )}^{2} \qquad ( 11 b )$$

$${w}_{w , v} = 1 - {( \frac{2 {R}_{0}}{{λ}_{0}} )}^{2} \qquad ( 11 c )$$

where w_{d,s} and w_{e,s}, respectively, are the weight fractions of drug and excipient in the solid fiber. Using the non-limiting parameter values was w_{d,s}˜0.87, w_{e,s}˜0.13, and R₀/λ₀=0.35, by Eqs. (11a)-(11c) the weight fractions of drug, excipient, and water in the viscous suspension, w_{d,s}˜0.43, w_{e,v}˜0.06, and w_{w,v}˜0.51.

Similarly, the concentration of excipient in the viscous excipient-water solution between the drug particles may be estimated by:

$${c}_{e , sol} = {ρ}_{sol} \frac{{w}_{e , v}}{{w}_{e , v} + {w}_{w , v}} \qquad ( 12 )$$

where ρₛₒₗ is the density of the viscous solution. Rough estimates of the concentrations and corresponding viscosities of the viscous excipient-water solutions between the drug particles are listed below in Table 1.

TABLE 1
Concentrations and viscosities of viscous excipient-
water solutions between drug particles.

| | M_{w} | c_{e, sol} | cₑ* | | μₛₒₗ | |
|---|---|---|---|---|---|---|
| | (kg/mol) | (mg/ml) | (mg/ml) | c_{e, sol}/cₑ* | (Pa · s) | μₛₒₗ/μ_{w} |
| A | 10 | 111 | 62 | 1.8 | 0.2 | 2.0 × 10² |
| B | 26 | 111 | 20 | 5.6 | 15.2 | 1.5 × 10⁴ |
| C | 86 | 111 | 6 | 18.5 | 617.9 | 6.2 × 10⁵ |

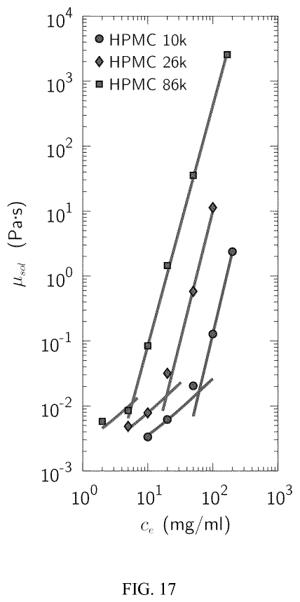

cₑ* and μₛₒₗ are obtained from the viscosity-concentration data of FIG. 17 and experimental example 5 presented later.
μ_{w} is the viscosity of water, 0.001 Pa · s.

For all three non-limiting dosage forms modeled herein, c_{e,sol}≈111 g/ml, Table 1. The viscosities of the solutions, however, are vastly different. For the 10k excipient (dosage form A), c_{e,sol} is of the order of cₑ*, and the viscosity, μₛₒₗ is only 200 times that of water, Table 1. For the 86k excipient (dosage form C), c_{e,sol}/cₑ*≈20, and μₛₒₗ is almost six orders of magnitude greater than that of water, Table 1.

### (g) Deformation and Fragmentation

If the viscosity of the viscous solution is small enough, the viscous fibers and dosage form may deform and fragment due to the capillary pressure in the pores and due to other forces, FIG. 6a. If the viscosity is too large, however, the dosage form may not fragment, and a viscous mass may be formed instead, FIG. 6b.

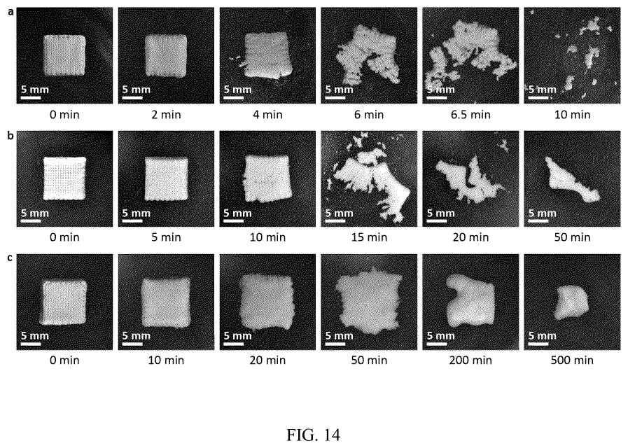

An in-depth analysis of dosage form fragmentation is far beyond the scope of this disclosure. Therefore, it may be better to focus on the role of cₑₛₒₗ/cₑ* in fragmentation. From the experimental results shown in FIGS. 14a-14c, the dosage form with the 10k excipient (c_{e,sol}/cₑ*=1.8) fragmented rapidly, that with the 26k excipient (c_{e,sol}/cₑ*=5.6) fragmented slowly, and that with the 86k excipient (c_{e,sol}/cₑ*=18.5) did not fragment at all, but formed a highly viscous mass. Thus, if c_{e,sol} is smaller than about 2 times cₑ*, the viscosity of the excipient-water solution surrounding the drug particles may be so small that the suspension easily fragments. If c_{e,sol}>>2-6 times cₑ*, however, the viscosity may be too large; an unfragmented, monolithic viscous mass may be formed instead. The behavior of dosage forms with c_{e,sol}˜2-6 times cₑ* may be intermediate.

Thus, by varying the excipient molecular weight (e.g., the disentanglement concentration of the excipient, cₑ*) and the concentration of excipient in the viscous excipient-water solution between the drug particles, c_{e,sol}, the fragmentation rate of the viscous drug-excipient-dissolution fluid suspension may be tailored. For achieving rapid fragmentation, c_{e,sol} may not exceed about 2-6 times cₑ*.

More models or concepts of fragmentation of a viscous suspension would be obvious to a person of ordinary skill in the art. All of them are within the spirit and scope of this disclosure.

### (h) Erosion of the Thick Viscous Suspension by Convection

At any time after immersion, the dosage forms may erode into the dissolution fluid by convective mass transfer. Moreover, both the low-viscosity fragments and the high-viscosity mass erode by convective mass transfer from the surface into the dissolution fluid. If the solubility of the excipient is far greater than that of the drug, the erosion rate of the viscous drug-excipient-water suspension is essentially determined by that of the viscous excipient-water solution between the drug particles. Adapting the prior work (see, e.g., A. H. Blaesi, N. Saka, Microstructural effects in drug release by solid and cellular polymeric dosage forms: A comparative study, Mater. Sci. Eng. C 80 (2017) 715-727; or the co-pending U.S. application Ser. No. 15/482,776 titled “Fibrous dosage form”), the erosion rate, dE/dt, of a viscous excipient-water solution in the flow field of a rotating, solid disk may be written as:

$$dE / dt = 0.62 ( \frac{{c}_{e}^{*}}{{c}_{e , s o l}} ) {( \frac{{μ}_{w}}{{D}_{e} {ρ}_{w}} )}^{1 / 3} {( \frac{{D}_{e}^{2} {ρ}_{w} Ω}{{μ}_{w}} )}^{1 / 2} \qquad ( 13 )$$

where cₑ* is the disentanglement concentration of the excipient, Dₑ its diffusivity, μ_{w} and ρ_{w} the viscosity and density of the dissolution fluid, and Ω the angular velocity of the paddle (or stirrer).

Substituting the non-limiting values of cₑ* from Table 1 and the non-limiting parameter values c_{e,sol}˜111 g/ml, μ_{w}˜0.001 Pa·s, Dₑ˜1.5×10⁻¹⁰ m²/s (HPMC 10k), 9×10⁻¹¹ m²/s (HPMC 26k), and 7.5×10⁻¹¹ m²/s (HPMC 86k), ρ_{w}˜1000 kg/m³, and Ω˜50 rpm, the erosion rate of the viscous solution with 10k excipient is about 2.25 μm/s, that with 26k excipient about 0.6 μm/s, and that with 86k excipient about 0.14 μm/s.

The time to erode a viscous fragment, or mass, of thickness Hᵥ eroding from one side may be estimated by:

$${t}_{e r} = \frac{{H}_{v}}{dE / dt} \qquad ( 14 )$$

In the rapidly fragmenting dosage form A, Hᵥ, may be ever decreasing, and thus the erosion time may be small. This hypothesis was supported by the experiments, FIG. 14: due to their small thickness and large surface area-to-volume ratio the fragments eroded rapidly after the dosage form fell apart.

By contrast, dosage form C may not fragment. Instead it may form a thick viscous mass; hence Hᵥ may be about the thickness of the dosage form. Using Hᵥ≈3 mm and the above erosion rate, by Eq. (17) tₑᵣ≈365 min. The experimental result shown in FIG. 14 is about 1.6 times longer than this estimate. This is reasonable considering that the effect of drug particles (and other effects) on the erosion rate was neglected.

Dosage form B may fragment somewhat and/or slowly, and it may not be possible to determine the “initial” thickness of the fragments. As expected, however, in the experiments shown in FIG. 14 the erosion time of dosage form B was between that of dosage form A and that of dosage form C.

More models of erosion of a viscous suspension would be obvious to a person of ordinary skill in the art. All of them are within the spirit and scope of this disclosure.

### (i) Drug Dissolution Time

As the excipient erodes the drug particles may be released from the dosage form or the drug-excipient-dissolution fluid suspension into the dissolution fluid. The released particles may then dissolve. If the particles are fairly small and fairly soluble in the dissolution fluid, dissolution of the particles may be fast.

Thus, in the case of the HPMC 10k dosage form (dosage form A), which may fragment extensively after its fibers are penetrated by water, the penetration time may be considered rate-limiting. Thus, the time to dissolve eighty percent of the drug content of the HPMC 10k dosage form (dosage form A) may be approximately:

t_{0.8}≅tₚₑₙ (16)

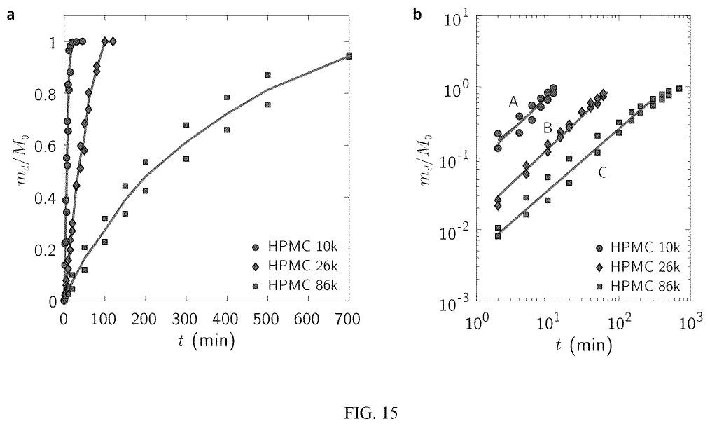

Using the values above, the calculated t_{0.8} time is 6 min. This reasonably agrees with the experimental results, thus validating the model, Table 2 and FIG. 15 later.

In the case of the HPMC 86k dosage form (dosage form C), however, the erosion time may be longest. Hence

t_{0.8}≅0.8tₑᵣ (17)

Using the above erosion time, the calculated t_{0.8} time is 292 min. Considering the vast assumptions that have been made, this again reasonably agrees with the experimental result, t_{0.8}=487 min, Table 2 and FIG. 15 later.

In case of the dosage form with HPMC 26k excipient (dosage form B), however, the fragment thickness, Hᵥ, may not be known; thus an empirical approach may be followed to determine t_{0.8}.

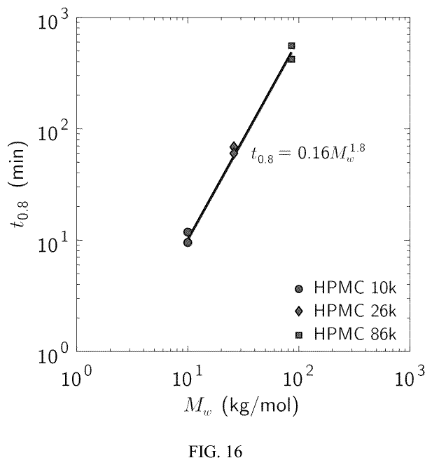

From the experimental results shown later in FIG. 16, the t_{0.8} time of the dosage forms increased with excipient molecular weight as t_{0.8}=0.16×M_{w}^{1.8} min, or approximately as the square of the molecular weight, in the range 10 kg/mol≤M≤86 kg/mol.

More models for estimating drug dissolution time of dosage forms as disclosed herein would be obvious to a person of ordinary skill in the art. All of them are within the spirit and scope of this disclosure.

### (j) Summary of Models

Models suggest that dissolution fluid percolates contiguous void space, and a capillary pressure develops in the pores of the fibers with an open and a closed end. The fluid may then diffuse through the thin fibers, and they may transition to a viscous suspension of drug particles, excipient, and dissolution fluid. If the viscosity of the suspension is low (as with HPMC 10k excipient), it may rapidly fragment due to the capillary pressure, and dissolve. The drug release time may then be essentially the time for diffusion through the fiber. If the viscosity of the suspension is high (as with the HPMC 86k excipient), however, a thick viscous mass may be formed, and the drug release time may be the time to erode the thick mass.

Between these extremes, the dosage form may fragment to some degree, and the t_{0.8} time may be determined by the time to erode the fragments. However, because the fragment thickness is not known precisely, an empirical approach may need to be followed. From data the t_{0.8} time increased with excipient molecular weight as t_{0.8}=0.16×M_{w}^{1.8} min, or approximately as the square of the molecular weight in the range 10-86 kg/mol.

In conclusion, the above models suggest that drug release by close-packed fibrous dosage forms with large drug loading and a single, biologically inert excipient can be appropriately tailored by varying the molecular weight of a hydroxypropyl methylcellulose excipient.

### Embodiments of the Dosage Form

In view of the design considerations, theoretical models and non-limiting examples above, which are suggestive and approximate rather than exact, the dosage forms disclosed herein may further comprise the following embodiments.

### (a) Geometry of Drug-Containing Solid and Three Dimensional Structural Framework

In some embodiments dissolution fluid may percolate into the interior of the structure (e.g., into at least one free space or into the free spaces) if the drug-containing solid comprises at least a continuous channel or free space having at least one, and preferably at least two openings in contact with said fluid. The more such channels exist with at least one, and preferably at least two ends in contact with a dissolution fluid the more uniformly may the structure be percolated. Uniform percolation generally is desirable in the invention herein.

Thus, in the invention herein a plurality of adjacent free spaces may combine to define one or more interconnected free spaces (e.g., free spaces that are “contiguous” or “in direct contact” or “merged” or “without any wall in between”) that may extend over a length at least half the thickness of the drug-containing solid. This includes, but is not limited to a plurality of adjacent free spaces combining to define one or more interconnected free spaces that extends over a length at least two thirds the thickness of the drug-containing solid, or over a length at least equal to the thickness of the drug-containing solid, or over a length at least equal to the side length of the drug-containing solid, or over a length and width at least equal to half the thickness of the drug-containing solid, or over a length and width at least equal to the thickness of the drug-containing solid, or over a length, width, and thickness at least equal to half the thickness of the drug-containing solid, or over the entire length, width, and thickness of the drug-containing solid.

Also, in some embodiments an interconnected free space comprises or occupies at least 30 percent (e.g., at least 40 percent, or at least 50 percent, or at least 60 percent, or at least 70 percent, or at least 80 percent, or 100 percent) of the free space of the drug-containing solid (e.g., at least 30 percent, or at least 40 percent, or at least 50 percent, or at least 60 percent, or at least 70 percent, or at least 80 percent, or 100 percent of the free space of the drug-containing solid are part of the same interconnected free space).

In preferred embodiments, all free spaces are interconnected forming a continuous, single open space. In the invention herein, if all free spaces of a drug-containing solid are interconnected the free space of said drug-containing solid is also referred to as “contiguous”. In drug-containing solids with contiguous free space, no walls (e.g., walls comprising the three dimensional structural framework of elements) must be ruptured to obtain an interconnected cluster of free space (e.g., an open channel of free space) from the outer surface of the drug-containing solid to a point (or to any point) in the free space within the internal structure. The entire free space or essentially all free spaces is/are accessible from (e.g., connected to) the outer surface of the drug-containing solid.

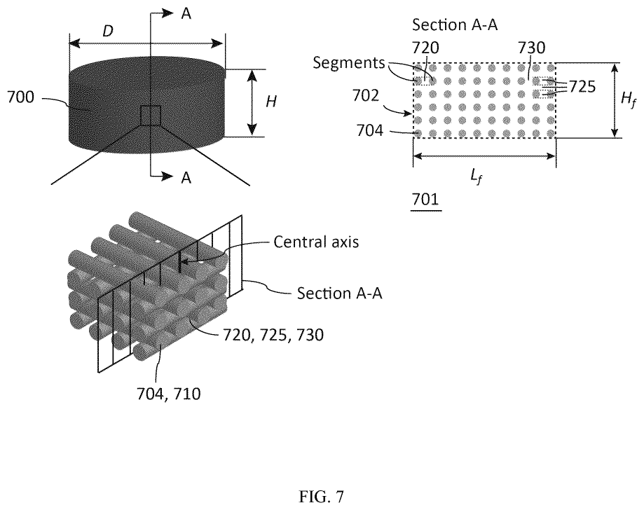

FIG. 7 schematically illustrates a pharmaceutical dosage form 700 comprising a drug-containing solid 701 having an outer surface 702 and an internal three dimensional structural framework 704 comprising a plurality of criss-crossed stacked layers of one or more fibrous structural elements 710. Said framework 704 is contiguous with and terminates at said outer surface 702. The fibrous structural elements 710 further have segments spaced apart from adjoining segments, thereby defining free spaces 720. A plurality of adjacent free spaces 725 combine to define at least one interconnected free space 730.

As shown in the non-limiting schematic of section A-A, said interconnected free space 730 extends over the entire length and thickness of the drug-containing solid 701 or the dosage form 700. In other words, the length, L_{f}, over which the interconnected free space 730 extends is the same as the length or diameter, D, of the dosage form 700 or drug-containing solid 701; the thickness, H_{f}, over which the interconnected free space 730 extends is the same as the thickness, H, of the dosage form 700 or drug-containing solid 701. It may be noted that the term “section” is understood herein as “plane” or “surface”. Thus a “section” is not a “projection” or “projected view”.

Moreover, in the non-limiting example of FIG. 7 the microstructure is rotationally symmetric. If the plane or section A-A is rotated by 90 degrees about the central axis the microstructure (e.g., the microstructural details) is/are the same. Thus, the interconnected free space 730 also extends over the entire width of the drug-containing solid 701 or the dosage form 700. In other words, the width over which the interconnected free space 730 extends is the same as the length or diameter, D, of the dosage form 700 or drug-containing solid 701.

Furthermore, in the non-limiting microstructure of FIG. 7, as shown in section A-A the interconnected free space 730 or free space 720 or free spaces 725 is/are contiguous. No walls (e.g., walls comprising the three dimensional structural framework 704 of elements) must be ruptured to obtain an interconnected cluster of free spaces (e.g., an open channel of free space) from the outer surface 702 of the drug-containing solid 701 to a point (or to any point or position) in the free space 720, 725, 730. Also, no walls (e.g., walls comprising the three dimensional structural framework 704 of elements) must be ruptured to obtain an interconnected cluster of free space (e.g., an open channel of free space) from any point or position within the free space 720, 725, 730 to any other point or position in the free space 720, 725, 730. The entire free space 720, 725, 730 is accessible from the outer surface 702 of the drug-containing solid 701. In addition, the entire free space 720, 725, 730 is accessible from any point, location, or position within the free space 720, 725, 730.

Additionally, the structure shown in FIG. 7 comprises fibers in a layer that are aligned unidirectionally (e.g., parallel). The fibers in the layers above and below said layer are aligned unidirectionally, too, and are aligned orthogonally to said layer (e.g., the fibers in the the layers above and below said layer are aligned orthogonally to the fibers in said layer, and vice versa). The fibers in the layers above and below said layer further touch or “merge with” fibers in said layer at inter-fiber contacts.

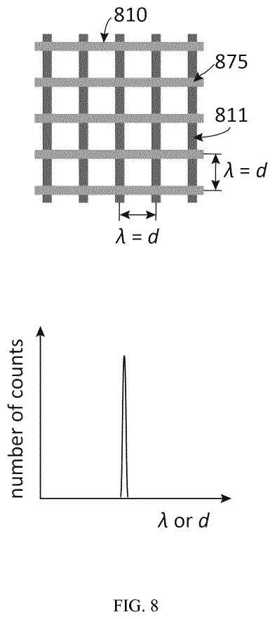

Several microstructural features can be defined to further characterize such structures. By way of example but not by way of limitation, as shown in FIG. 8, the structural framework 810 may be considered a network comprising nodes or vertices at the inter-fiber contacts 875 and edges 811 defined by the fiber segments of length, λ, between adjacent nodes or vertices 875.

FIG. 8 also shows a histogram of the length, λ, of fiber segments between adjacent point contacts. The λ values in this non-limiting example are distributed in a very narrow window or zone around the average, λ_{avg}. Thus, the standard deviation of the λ values is very small; λ is precisely controlled; the structure is repeatable, regular, deterministic, and ordered.

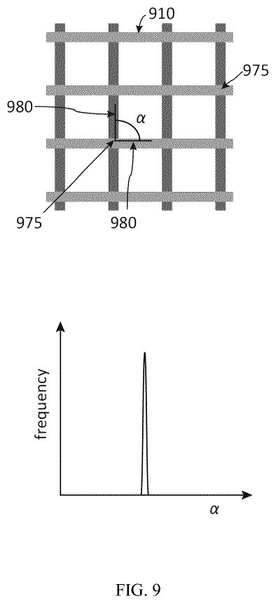

Similarly, as shown in FIG. 9, at the inter-fiber contacts 975 the two tangents of two contacting fibers or fiber segments 980, may form an angle, α. FIG. 9 also shows a histogram of the contact angle, α, across the three dimensional structural framework 910. Also the α values in this non-limiting example are distributed in a very narrow window or zone around the average, α_{avg}. Thus, the standard deviation of the α values is very small; α is precisely controlled; the structure is repeatable, regular, deterministic, and ordered.

More examples of fibrous structures according to the invention herein would be obvious to a person of ordinary skill in the art. All of them are within the scope of this disclosure. Furthermore, many of the above features and characteristics may also apply to (e.g., the features or characteristics may be similar to the features or characteristics of) three-dimensional structural frameworks of stacked layers of sheets, or stacked layers of beads (or particles), as shown, for example, in the co-pending International Application No. PCT/US2019/052030 filed on Sep. 19, 2019, and titled “Dosage form comprising structured solid-solution framework of sparingly-soluble drug and method for manufacture thereof”. Such features or characteristics are obvious to a person of ordinary skill in the art who is given all information disclosed in this specification. Application of such features or characteristics to three-dimensional structural frameworks of stacked layers of beads (or particles), or stacked layers sheets (or two-dimensional elements), is included in the invention herein.

Any more microstructures of three dimensional structural frameworks of elements or fibers would be obvious to a person of ordinary skill in the art. All of them are included in this invention.

### (b) Free Spacing Between Elements

Typically, moreover, for dissolution fluid to percolate into the interior of the structure the channel size or diameter (e.g., channel width, or pore size, or free spacing, or effective free spacing) between elements or segments must be on the micro- or macro-scale. Thus, in some embodiments, the effective free spacing, λ_{f,e}, between elements (e.g., fibers) or segments across one or more connected free spaces (e.g., the channel size or channel diameter) is greater than 1 μm. This includes, but is not limited to λ_{f,e} greater than 1.25 μm, or greater than 1.5 μm, or greater than 1.75 μm, or greater than 2 μm, or greater than 5 μm, or greater than 7 μm, or greater than 10 μm, or greater than 15 μm, or greater than 20 μm, or greater than 25 μm, or greater than 30 μm, or greater than 40 μm, or greater than 50 μm.

Because the dosage form volume is generally limited, however, the drug and excipient masses that can be loaded in the dosage form may be too small if the effective free spacing is too large. Thus, in some embodiments, the effective free spacing across an interconnected free space may be in the ranges 1 μm-5 mm, 1 μm-3 mm, 1.25 μm-5 mm, 1.5 μm-5 mm, 1.5 μm-3 mm, 5 μm-2.5 mm, 10 μm-2 mm, 10 μm-4 mm, 5 μm-4 mm, 10 μm-3 mm, 15 μm-3 mm, 20 μm-3 mm, 30 μm-4 mm, 40 μm-4 mm, or 50 μm-4 mm.

In some embodiments, moreover, the effective free spacing between segments or elements across the one or more free spaces (e.g., across all free spaces of the dosage form) is in the range 1 μm-3 mm. This includes, but is not limited to an effective free spacing between segments or elements across the one or more free spaces in the ranges 1 μm-2.5 mm, or 1 μm-2 mm, or 2 μm-3 mm, or 2 μm-2.5 mm, or 5 μm-3 mm, or 5 μm-2.5 mm, or 10 μm-3 mm, or 10 μm-2.5 mm, or 15 μm-3 mm, or 15 μm-2.5 mm, or 20 μm-3 mm, or 20 μm-2.5 mm. The effective free spacing may be determined experimentally from microstructural images (e.g., scanning electron micrographs, micro computed tomography scans, and so on) of the drug-containing solid. Non-limiting examples describing and illustrating how an effective free spacing may be determined from microstructural images are described and illustrated in the U.S. application Ser. No. 15/482,776 titled “Fibrous dosage form”.

It may be noted, moreover, that in some embodiments herein the free spacing or effective free spacing between elements or segments across the three dimensional structural framework or across one or more open pore networks is precisely controlled.

Furthermore, the free spacing between elements and the surface composition of elements are generally designed to enable percolation of physiological, body, or dissolution fluid into the dosage form structure upon immersion of the dosage form in said fluid. Thus, in some embodiments the free spacing between segments and the composition of the surface of the one or more elements are so that the percolation time of physiological/body fluid into one or more interconnected free spaces of the drug-containing solid is no greater than 300 seconds under physiological conditions. This includes, but is not limited to a percolation time of physiological/body fluid into one or more interconnected free spaces of the drug-containing solid no greater than 200, or no greater than 100 seconds, or no greater than 50 seconds, or no greater than 20 seconds, or no greater than 10 seconds under physiological conditions.

In addition, in some embodiments, upon immersion of the drug-containing solid in a physiological fluid, said fluid percolates more than 40 percent of the free spaces of said drug-containing solid in no more than 600 seconds of immersion.

In some embodiments, moreover, upon immersion of the drug-containing solid in a physiological fluid, said fluid percolates more than 60 percent of the free spaces of said drug-containing solid in no more than 400 seconds of immersion.

In some embodiments, furthermore, upon immersion of the drug-containing solid in a physiological fluid, said fluid percolates more than 50 percent of the free spaces of said drug-containing solid in no more than 200 seconds of immersion.

In some embodiments, furthermore, upon immersion of the drug-containing solid in a physiological fluid, said fluid percolates more than 40 percent of the free spaces of said drug-containing solid in no more than 100 seconds of immersion.

Any more details of free spacings would be obvious to a person of ordinary skill in the art. All of them are included in this invention.

### (c) Composition of Free Space

Generally, one or more free spaces (e.g., one or more interconnected free spaces) are filled with a matter that is removable by a physiological fluid under physiological conditions. Such matter that is removable by a physiological fluid under physiological conditions can, for example, be a gas which escapes a free space upon percolation of said free space by said physiological fluid. Such matter that is removable by a physiological fluid under physiological conditions can, however, also be a solid that is highly soluble in said physiological fluid, and thus dissolves rapidly upon contact with or immersion in said physiological fluid.

Non-limiting examples of biocompatible gases that may fill free space include air, nitrogen, CO₂, argon, oxygen, and nitric oxide, among others.

Non-limiting examples of solids that are removed or dissolved after contact with physiological/body fluid include sugars or polyols, such as Sucrose, Fructose, Galactose, Lactose, Maltose, Glucose, Maltodextrin, Mannitol, Maltitol, Isomalt, Lactitol, Xylitol, Sorbitol, among others. Other examples of solids include polymers, such as polyethylene glycol, polyvinyl pyrrolidone, polyvinyl alcohol, among others. Typically, a solid material should have a solubility in physiological/body fluid (e.g., an aqueous physiological or body fluid) under physiological conditions greater than 50 g/l to be removed or dissolved rapidly after contact with dissolution medium. This includes, but is not limited to a solubility greater than 75 g/l, or greater than 100 g/l, or greater than 150 g/l, or greater than 200 g/l. The diffusivity of the solid material (as dissolved molecule in physiological/body fluid under physiological conditions) should typically be greater than 4×10⁻¹² m²/s if the solid material must be dissolved rapidly after contact with dissolution medium. This includes, but is not limited to a diffusivity in physiological fluid under physiological conditions greater than 6×10⁻¹² m²/s, or greater than 8×10⁻¹² m²/s, or greater than 1×10⁻¹¹ m²/s, or greater than 2×10⁻¹¹ m²/s, or greater than 5×10⁻¹¹ m²/s.

In some embodiments, moreover, a solid that may fill free space has a molecular weight (e.g., average molecular weight, such as number average molecular weight or weight average molecular weight) no greater than 80 kg/mol. This includes, but is not limited to a molecular weight (e.g., average molecular weight, such as number average molecular weight or weight average molecular weight) no greater than 70 kg/mol, or no greater than 60 kg/mol, or no greater than 50 kg/mol, or no greater than 45 kg/mol, or no greater than 40 kg/mol, or no greater than 35 kg/mol, or no greater than 30 kg/mol.

Further compositions of free space obvious to a person of ordinary skill in the art who is given all information of this specification are all included in this invention.

### (d) Geometry of Elements

After percolation of free space or one or more interconnected free spaces, dissolution fluid or physiological fluid may surround one or more elements (e.g., fibers) or segments thereof. For achieving a large specific surface area (i.e., a large surface area-to-volume ratio) of elements in contact with dissolution fluid, in some embodiments the one or more elements (e.g., fibers) have an average thickness, h₀, no greater than 2.5 mm. This includes, but is not limited to h₀ no greater than 2 mm, or no greater than 1.75 mm, or no greater than 1.5 mm, or no greater than 1.25 mm, or no greater than 1 mm, or no greater than 750 μm.

It may be noted, however, that if the elements are very thin and tightly packed, the spacing and free spacing between the elements can be so small that the rate at which dissolution fluid percolates or flows into the free space is limited. Furthermore, dosage forms with very thin elements may be difficult to manufacture by, for example, 3D-micro-patterning. Thus, in some embodiments the one or more elements (e.g., fibers) have an average thickness, h₀, in the ranges of 10 μm-2 mm. This includes, but is not limited to average thickness, h₀, of one or more elements (e.g., fibers) in the ranges 15 μm-2 mm, 20 μm-2 mm, 25 μm-2 mm, 30 μm-2 mm, 20 μm-1.5 mm, 25 μm-1.5 mm, 25 μm-1.25 mm, 25 μm-1 mm, 30 μm-1.5 mm, 30 μm-1.25 mm, 30 μm-1 μm, 40 μm-1.5 mm, or 50 μm-1.25 mm.

In some embodiments, moreover, the average thickness of the one or more elements (e.g., fibers) comprising or composing (e.g., producing, making up, etc.) the three dimensional structural network (e.g., the average thickness of the elements in the three dimensional structural network) is precisely controlled.

The element or fiber thickness, h, may be considered the smallest dimension of an element (i.e., h≤w and h≤l, where h, w and l are the thickness, width and length of the element, respectively). The average element or fiber thickness, h₀, is the average of the element or fiber thickness along the length of the one or more elements or fibers. A non-limiting example illustrating how an average fiber thickness may be derived is presented in U.S. application Ser. No. 15/482,776 titled “Fibrous dosage form”.

In some embodiments, at least one outer surface of one or more elements (e.g., the outer surface or one or more fibers or the outer surface of a fiber segment) comprises a coating. Said coating may cover part of or the entire outer surface of one or more elements or a segment thereof. Said coating may further have a composition that is different from the composition of one or more elements or a segment thereof. The coating may be a solid, and may or may not comprise or contain a drug.

### (e) Microstructure and Composition of Elements

Generally, one or more elements (e.g., fibers) or segments thereof may comprise a continuous (e.g., a single, or internally connected) solid matrix through their thickness. In other words, one or more elements (e.g., one or more fibers) may comprise an outer surface and an internal, continuous solid matrix that is contiguous with and terminating at said outer surface.

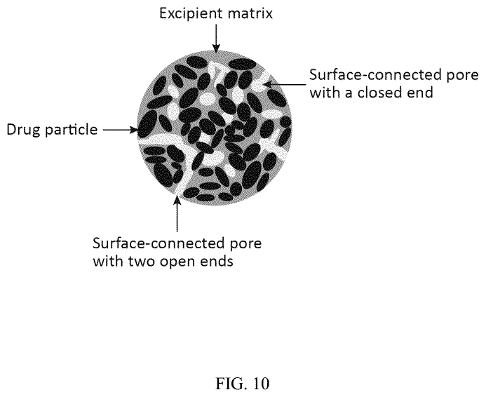

FIG. 10 presents a non-limiting example of a microstructure of an element (e.g., a fiber) according to the invention herein. The element (e.g., fiber) comprises an excipient matrix, drug particles, and one or more pores with a composition distinct from the composition of the excipient matrix and the drug particles through its thickness.

The excipient matrix may bind and carry said drug particles and pores. Thus the excipient matrix may be substantially connected through the thickness of the element. Generally, the composition of the excipient matrix comprises at least a polymer that is soluble in a relevant physiological fluid (e.g., at least a physiological fluid-soluble polymeric excipient, or one or more physiological fluid-soluble polymeric excipients).

In some embodiments, a dosage form should disintegrate rapidly upon immersion in a physiological fluid. Thus, to ensure that the concentration of physiological fluid-soluble polymeric excipient in a viscous excipient-dissolution fluid solution is small and the dosage form deforms or fragments after transitioning to viscous, in some embodiments the weight fraction of physiological fluid-soluble polymeric excipient in an element (e.g., the weight fraction of physiological fluid-soluble polymeric excipient in a fiber, or the weight fraction of physiological fluid-soluble polymeric excipient in a framework of elements or fibers, etc.) is in the range of 0.04-0.4. This includes, but is not limited the weight fraction of physiological fluid-soluble polymeric excipient in an element (e.g., the weight fraction of physiological fluid-soluble polymeric excipient in a fiber, or the weight fraction of physiological fluid-soluble polymeric excipient in a framework of elements or fibers, etc.) in the ranges 0.05-0.4, or 0.05-0.35, or 0.05-0.3, or 0.05-0.25.

Similarly, in some embodiments the volume fraction of physiological fluid-soluble polymeric excipient in an element (e.g., the volume fraction of physiological fluid-soluble polymeric excipient in a fiber, or the volume fraction of physiological fluid-soluble polymeric excipient in a framework of elements or fibers, etc.) is in the range of 0.04-0.4. This includes, but is not limited a volume fraction of physiological fluid-soluble polymeric excipient in an element (e.g., a volume fraction of physiological fluid-soluble polymeric excipient in a fiber, or a volume fraction of physiological fluid-soluble polymeric excipient in a framework of elements or fibers, etc.) in the ranges 0.05-0.4, or 0.05-0.35, or 0.05-0.3, or 0.05-0.25.

In some embodiments, moreover, the mass of physiological fluid-soluble polymeric excipient in the drug-containing solid divided by the volume of free space in the drug-containing solid is no greater than six times the disentanglement concentration of said physiological fluid-soluble polymeric excipient in said physiological fluid.

In some embodiments, furthermore, physiological fluid-soluble polymeric excipient is substantially uniformly distributed across the excipient matrix. It may be noted that in the invention herein, the concentration of a substance may generally be uniform across a region of the structural network if the standard deviation of multiple (e.g., multiple, randomly selected, e.g., at least three or at least 4 or at least 5 or at least 6 or at least 10 or at least 20 randomly selected) concentration samples from said region is less than the average concentration. This includes, but is not limited to a standard deviation of multiple (e.g., multiple, randomly selected, e.g., at least three or at least 4 or at least 5 or at least 6 or at least 10 or at least 20 randomly selected) concentration samples from said region less than half, or less than one third, or less than a quarter, or less than one fifth, or less than one sixth, or less than one eight, or less than one tenth, or less than one fifteenth of the average concentration.

The drug particles generally comprise substantially discrete and/or substantially disconnected portions or units of matter distributed or dispersed across the thickness of an element. The composition of the drug particles generally comprises at least an active ingredient. This includes, but is not limited to particles that predominantly consist of active ingredient, such as particles having an active ingredient weight fraction greater than 0.9, or greater than 0.95, or greater than 0.98, etc. This also includes, but is not limited to particles that include one or more active ingredients and one or more excipients. By way of example but not by way of limitation, in a drug particle one or more active ingredients may be dispersed or dissolved in one more more excipients. In this case, the weight or volume fraction of active ingredient (e.g., the weight or volume fraction of drug) in a drug particle can be substantially smaller than one (e.g., between zero and one).

In some embodiments, the volume fraction of drug particles in an element (or in one or more elements, such as one or more fibers) is greater than 0.5.

Similarly, in some embodiments the weight fraction of drug particles in an element (or in one or more elements, such as one or more fibers) is greater than 0.5.

In some embodiments, moreover, the drug particles or molecules are uniformly (e.g., spatially uniformly) or almost uniformly dispersed across or through the fiber thickness or along the fiber length, or even through or across the three dimensional structural network of fibers.

Also, in some embodiments the weight fraction of drug (e.g., the weight fraction of drug in the form of drug molecules and/or drug particles) in an element, or in a three-dimensional structural framework of elements, or in a drug-containing solid is in the range of 0.45 to 0.95. This includes, but is not limited to a drug weight fraction in an element, or in a three-dimensional structural framework of elements, or in a drug-containing solid in the ranges 0.5-0.95, 0.55-0.95, or 0.6-0.95, or 0.65-0.95, or 0.7-0.95, or 0.75-0.95, or 0.8-0.95.

The one or more pores generally comprise small openings or interstices within one or more elements (e.g., within one or more fibers). Generally, the one or more pores can be connected or disconnected across the thickness of an element. They can further be surface-connected and open, or they can be internal and closed. A surface-connected pore generally is open and connected to or in contact with the surface of an element (or with its coating). A surface-connected pore can have at least two open ends, or it can have one open end and one or more closed ends. In some embodiments, therefore, an element (e.g., a fiber) may comprise at least a surface-connected pore.

For achieving rapid dissolution of an element or dosage form upon immersion of said element or dosage form in a physiological fluid, it is desirable that the content or composition of pores is removable by said physiological fluid under physiological conditions. Thus, by way of example but not by way of limitation, the composition of one or more pores may comprise at least a gas, such as air, nitrogen, oxygen, CO₂, and so on. The composition of one or more pores may also include, but is not limited to a solid that is highly soluble in a physiological fluid, such as sugars, polyols, highly water-soluble polymers, and so on.

In some embodiments the volume fraction of pores in an element (or in one or more elements, such as one or more fibers) is in the range 0.03-0.4. This includes, but is not limited to a volume fraction of pores in one or more elements in the ranges 0.01-0.35, 0.01-0.3, 0.02-0.4, 0.05-0.4, 0.06-0.4, 0.02-0.35, 0.05-0.35, 0.02-0.3, 0.05-0.3, or 0.05-0.25. The volume fraction of pores in an element is referred to herein as the volume of pores in an element divided by the volume of said element. It is also understood herein as the “porosity of an element”.

In some embodiments, furthermore, the pores in one or more elements comprise an average size (e.g., an average width or average diameter or average thickness along the pore length) in the range of 0.5 μm to 150 μm. This includes, but is not limited to an average pore size in the ranges 0.5 μm-125 μm, or 0.5 μm-100 μm, or 1 μm-150 μm, or 1 μm-125 μm, or 1 μm-100 μm, or 2 μm-150 μm, or 2 μm-125 μm, or 2 μm-100 μm.

Generally, moreover, within the above ranges of the average pore size, the pore size may be randomly or almost randomly distributed. Also, the pore locations in one or more fibers may be randomly or almost randomly distributed.

It may be noted that an element may further comprise additional features, compositions, or variants not described above. By way of example but not by way of limitation, any element may further comprise multiple active ingredients, particles of an excipient that is sparingly soluble or insoluble in a physiological fluid, and so on. Any such feature or variant obvious to a person of ordinary skill in the art is included in the spirit and scope of this invention.

### d) Properties and Examples of Physiological Fluid-Soluble Polymeric Excipient

For enabling dissolution fluid penetration into an excipient matrix or into an element (e.g., a fiber), in some embodiments a physiological fluid-soluble polymeric excipient may be absorptive of a physiological/body fluid under physiological conditions. In the invention herein, an excipient is absorptive of a physiological/body fluid if the effective diffusivity of physiological/body fluid in said excipient (and/or a fiber or a segment comprising said excipient) is greater than 0.1×10⁻¹¹ m²/s under physiological conditions. In other examples without limitation, the effective diffusivity of physiological/body fluid in an absorptive excipient (and/or a fiber or a segment comprising said excipient) may be greater than 0.2×10⁻¹¹ m²/s, greater than 0.5×10⁻¹¹ m²/s, greater than 1×10⁻¹¹ m²/s, or greater than 2×10⁻¹¹ m²/s under physiological conditions.

Alternatively, a rate of penetration may be specified. In some embodiments, the rate of penetration of a physiological/body fluid into a solid, absorptive excipient (and/or an element) is greater than an average thickness of the one or more drug-containing elements divided by 3600 seconds (i.e., h₀/3600 μm/s). In other examples without limitation, rate of penetration may be greater than h₀/1800 μm/s, greater than h₀/1200 μm/s, greater than h₀/800 μm/s, or greater than h₀/600 μm/s. Non-limiting examples for deriving the effective diffusivity or rate of penetration are, for example, presented in U.S. application Ser. No. 15/482,776 titled “Fibrous dosage form”.

Furthermore, to ensure that the deformed dosage form or fragmented parts of the dosage form dissolve, in some embodiments at least one physiological fluid-soluble polymeric excipient has a solubility greater than 0.1 g/l in physiological/body fluids under physiological conditions. This includes, but is not limited to a solubility of at least one physiological fluid-soluble polymeric excipient in a physiological/body fluid greater than 0.5 g/l, or greater than 1 g/l, or greater than 5 g/l, or greater than 10 g/l, or greater than 20 g/l, or greater than 30 g/l, or greater than 50 g/l, or greater than 70 g/l, or greater than 100 g/l.

For polymers that form viscous solutions when combined with a dissolution medium, the ‘solubility’ in the context of this invention is the polymer concentration in physiological/body fluid at which the average shear viscosity of the polymer-physiological/body fluid solution is 5 Pa·s in the shear rate range 1-100 l/s under physiological conditions. The pH value of the physiological/body fluid may thereby be adjusted to the specific physiological condition of interest. By contrast, for a material that does not form a viscous solution when combined with a dissolution medium, the solubility herein is the ratio of the maximum mass of said material that can be dissolved in a given volume of dissolution medium at equilibrium divided by said volume of the medium. It may, for example, be determined by optical methods.

In some embodiments herein, moreover, the diffusivity of a dissolved or solvated molecule of at least one physiological fluid-soluble polymeric excipient in a physiological/body fluid may be greater than 0.5×10⁻¹² m²/s under physiological conditions. This includes, but is not limited to a diffusivity of a dissolved molecule of at least one physiological fluid-soluble polymeric excipient in a physiological/body fluid greater than 1×10⁻¹² m²/s, or greater than 2×10⁻¹² m²/s, or greater than 4×10⁻¹² m²/s, or greater than 6×10⁻¹² m²/s, or greater than 8×10⁻¹² m²/s under physiological conditions.

Non-limiting examples of excipients that satisfy some or all the requirements of the physiological fluid-soluble polymeric excipient include hydroxypropyl methylcellulose, hydroxyethyl cellulose, polyvinyl alcohol, polyvinylpyrrolidone, hydroxypropyl methylcellulose acetate succinate, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), vinylpyrrolidone-vinyl acetate copolymer, carbopol (e.g., acrylic acid crosslinked with allyl sucrose or allyl pentaerythritol), among others.

In some embodiments, moreover, the molecular weight of at least one physiological fluid-soluble polymeric excipient is between 1 kg/mol and 100 kg/mol. This includes, but is not limited to a molecular weight of at least one water-soluble polymeric excipient in the ranges 1 kg/mol-90 kg/mol, 1 kg/mol-80 kg/mol, 2 kg/mol-70 kg/mol, 1 kg/mol-60 kg/mol, 2 kg/mol-80 kg/mol, 2 kg/mol-50 kg/mol, 2 kg/mol-40 kg/mol, or 2 kg/mol-30 kg/mol, or 4 kg/mol-50 kg/mol, or 5 kg/mol-50 kg/mol, or 4 kg/mol-40 kg/mol.

In some preferred embodiments, furthermore, at least one physiological fluid-soluble excipient comprises hydroxypropyl methylcellulose. The average molecular weight of said hydroxypropyl methylcellulose excipient may be in the range between 1 kg/mol and 80 kg/mol. Other non-limiting ranges of the average molecular weight of said hydroxypropyl methyl cellulose excipient are 1 kg/mol-70 kg/mol, or 1 kg/mol-60 kg/mol, or 1 kg/mol-50 kg/mol, or 1 kg/mol-40 kg/mol, or 2 kg/mol-50 kg/mol, or 2 kg/mol-40 kg/mol.

It may be obvious to a person of ordinary skill in the art that additional excipients having additional functionalities may be added to one or more fibers or the dosage form. Moreover, it would be obvious to a person of ordinary skill in the art that one excipient material (e.g., one constituent) may assume or have multiple functions or functionalities. All such excipients, excipient combinations, or additional functionalities obvious to a person of ordinary skill in the art are within the spirit and scope of this disclosure.

### e) Disintegration and Drug Release Properties of Drug-Containing Solid and Dosage Form

In some embodiments, upon immersion of the dosage form or drug-containing solid in a physiological or dissolution fluid under physiological conditions, said fluid may percolate one or more interconnected free spaces. The fluid may then surround one or more elements (e.g., fibers) or segments thereof, and percolate pores in the elements that are connected to the surface. Also, a capillary pressure may develop in surface-connected pores with at least a closed end.

Furthermore, as the fluid surrounds the solid elements (e.g., fibers) or segments thereof, the fluid may penetrate or diffuse into the elements or segments (e.g., into the physiological fluid-absorptive excipient in the elements or segments). Consequently, the elements (e.g., fibers) or segments surrounded by the dissolution fluid may transition to a viscous suspension comprising drug particles embedded in a viscous excipient-dissolution fluid solution. The viscous suspension (e.g., the viscous elements) may fragment due to the capillary pressure and other forces (e.g., shear forces, gravity, etc) applied by the dissolution fluid.

The surface area-to-volume ratio may be enhanced due to the small size of the viscous fragments. Thus the physiological fluid-soluble and/or physiological fluid-absorptive excipient in the fragments may erode or dissolve rapidly, thereby releasing drug molecules and drug particles. The size or diameter of the released drug particles may be fairly small. Thus, even if the solubility of the particles is fairly low, they may dissolve rapidly in the physiological or dissolution fluid.

Therefore, the above disintegration process, and the dosage forms disclosed herein, enable rapid release of drug particles (e.g., small drug particles) from highly drug-loaded, densely-packed structures, and promote rapid drug dissolution. The dosage forms herein are particularly useful for rapidly releasing and dissolving large quantities of drugs with fairly low aqueous solubility or fairly high hydrophobicity.

In some embodiments, accordingly, at least one active ingredient or drug comprises a solubility in an aqueous physiological or body fluid under physiological conditions no greater than 50 mg/ml. This includes, but is not limited to a solubility of least one active pharmaceutical ingredient or drug in an aqueous physiological or body fluid under physiological conditions no greater than 40 mg/ml, or no greater than 30 mg/ml, or no greater than 20 mg/ml, or no greater than 10 mg/ml, or no greater than 5 mg/ml, or no greater than 2 mg/ml, or no greater than 1 mg/ml, or no greater than 0.5 mg/ml.

In some embodiments, furthermore, the three-dimensional structural network of fibers or drug-containing solid or dosage form comprises a drug mass per unit volume greater than 0.45 mg/ml. This includes, but is not limited to a drug mass per unit volume greater than 0.5 mg/ml, or greater than 0.55 mg/ml, or greater than 0.6 mg/ml, or greater than 0.65 mg/ml, or greater than 0.7 mg/ml, or greater than 0.75 mg/ml, or greater than 0.8 mg/ml, or greater than 0.85 mg/ml, or in the ranges 0.45 mg/ml-5 mg/ml, 0.5 mg/ml-5 mg/ml, 0.55 mg/ml-5 mg/ml, 0.6 mg/ml-5 mg/ml, 0.65 mg/ml-5 mg/ml, 0.7 mg/ml-5 mg/ml, 0.75 mg/ml-5 mg/ml, 0.8 mg/ml-5 mg/ml, or 0.85 mg/ml-5 mg/ml.

In some embodiments, moreover, the three-dimensional structural network of fibers or drug-containing solid or dosage form comprises a drug mass greater than 50 mg. This includes, but is not limited to a drug mass greater than 75 mg, or greater than 100 mg, or greater than 125 mg, or greater than 150 mg, or greater than 175 mg, or greater than 200 mg, or greater than 225 mg, or greater than 250 mg, or greater than 275 mg, or greater than 300 mg, or in the ranges 50 mg-5 g, 75 mg-5 g, 100 mg-5 g, 125 mg-5 g, 150 mg-5 g, 175 mg-5 g, 200 mg-5 g, 225 mg-5 g, 250 mg-5 g, 275 mg-5 g, or 300 mg-5 g.

In some embodiments, furthermore, eighty percent of the drug content in the drug-containing solid is released (e.g., drug particles are released or drug molecules are released) from the drug-containing solid into a physiological fluid or body fluid under physiological conditions in less than 45 minutes after immersion of said drug-containing solid in said physiological or body fluid. This includes, but is not limited to a drug-containing solid that releases eighty percent of its drug content into a physiological or body fluid under physiological conditions in less than 40 minutes, or in less than 35 minutes, or in less than 30 minutes, or in less than 25 minutes, or in less than 20 minutes, or in less than 15 minutes, or in less than 10 minutes, or in 1-45 minutes, or in 1-30 minutes, or in 2-45 minutes, or in 2-30 minutes, or in 2-15 minutes after immersion of said drug-containing solid in said physiological or body fluid.

### Aspects of the Method to Manufacture Disclosed Dosage Forms

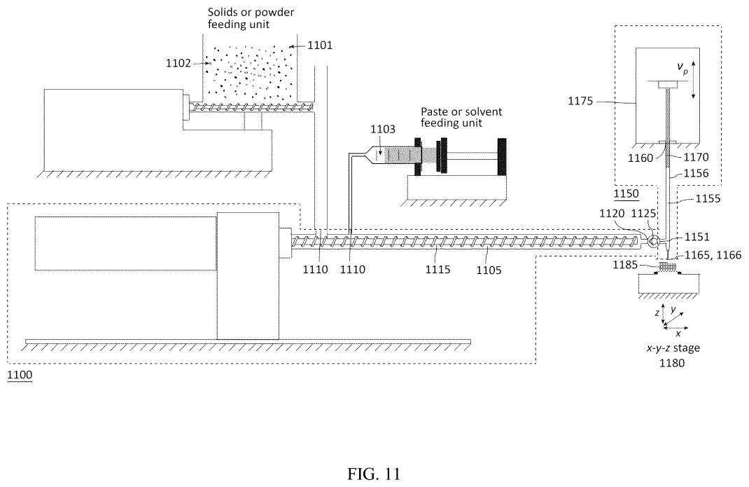

FIG. 11 presents a non-limiting example of a method of manufacturing pharmaceutical solid dosage forms according to this invention. At least one of each active ingredient 1101, excipient 1102, and solvent 1103 are injected into the extrusion channel 1105 of a first extruder 1100, said channel 1105 terminating at at least one exit port 1120. Said exit port 1120 having a valve 1125 (e.g., a check valve, etc.) mated to at least one input port 1151 of at least one second extruder 1150. In the first extruder's extrusion channel 1105 the injected active ingredient 1101, excipient 1102, and solvent 1103 are mixed to form a plasticized matrix. The plasticized matrix is conveyed to said first extruder's exit port 1120 and extruded through said exit port 1120 and the valve 1125, thereby filling at least one extrusion channel 1155 of at least one second extruder 1150 with said extruded plasticized matrix. The second extruder channel 1155 terminates at at least one fiber fabrication exit port 1166. The plasticized matrix in said second extruder extrusion channel 1155 is then extruded through said fiber fabrication port 1166 at a controlled speed by an advancing piston 1170. The extruded plasticized fiber is deposited onto a fiber assembling stage 1180 to form a three dimensional fiber structural framework 1185 defined by the motion of said stage 1180. The three dimensional fiber structural framework 1185 is then dried (e.g., the solvent is removed from the framework 285) to form a three dimensional structural framework of one or more repeatably arranged fibers.

For more examples of methods of manufacturing dosage forms as disclosed herein, see, e.g., the International Application No. PCT/US21/22860 filed on Mar. 17, 2021 and titled “Method and apparatus for 3D-micro-patterning”. It may be obvious to a person of ordinary skill in the art that many more examples of the method to manufacture the disclosed dosage form could be presented. Any more examples of methods to manufacture the dosage form disclosed obvious to a person of ordinary skill in the art are included in the scope of this invention.

### EXPERIMENTAL EXAMPLES

The following examples present ways by which dosage forms as disclosed herein may be prepared and analyzed, and will enable one of skill in the art to more readily understand the principle of the invention. The examples are presented by way of illustration and are not meant to be limiting in any way.

### Example 1: Preparation of Fibrous Dosage Forms by 3D-Printing

Three different dosage forms with the same drug but three different excipients were prepared and tested. The drug was ibuprofen and was received as solid particles with a diameter of about 20 μm. The three excipients, hydroxypropyl methylcellulose (HPMC) with molecular weights 10 kg/mol (HPMC 10k), 26 kg/mol (HPMC 26k), and 86 kg/mol (HPMC 86k), were also received as solid particles. Deionized water was used as the solvent for preparing the dosage forms.

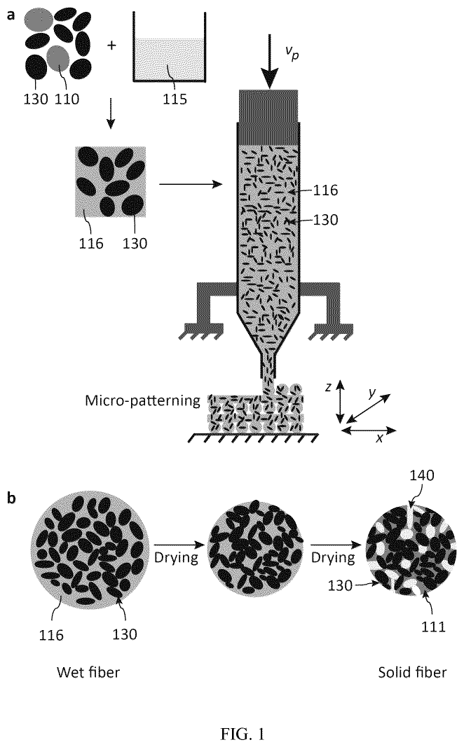

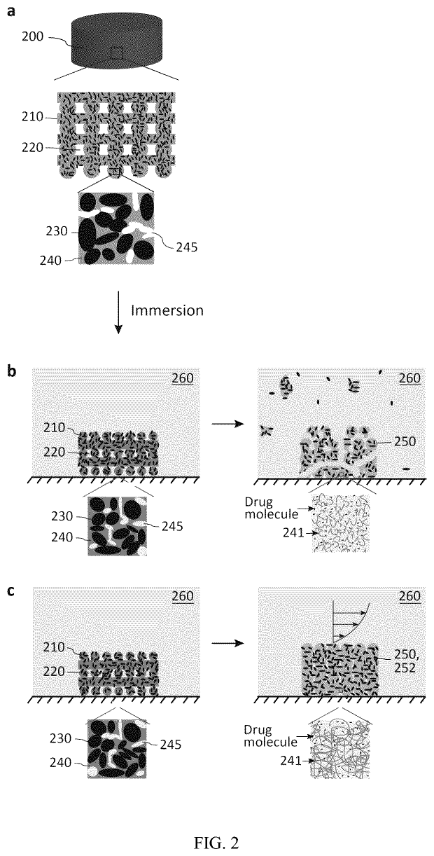

The dosage forms were prepared as illustrated schematically in FIG. 1. The excipient was first dissolved in deionized water to form a uniform viscous solution with an excipient weight fraction of 26 wt % for the HPMC 10k, 19 wt % for the HPMC 26k, and 11 wt % for the HPMC 86k excipient. Ibuprofen particles were then mixed with the water-excipient solution to form a uniform viscous paste. The paste was put in a syringe equipped with a hypodermic needle of inner radius, Rₙ=212 μm. It was then extruded through the needle and patterned as a fibrous dosage form with orthogonal cross-ply structure (e.g., as shown schematically in FIG. 1). The nominal inter-fiber spacing, λₙ (e.g., the inter-fiber spacing of the wet, patterned structure before drying), was 600 μm. While patterning and after, warm air at 30° C. and a velocity of about 1 m/s was blown over the dosage form to evaporate the water and solidify the structure. The solid dosage forms consisted of 87 wt % ibuprofen and 13 wt % excipient (HPMC 10k, dosage form A; HPMC 26k, dosage form B; and HPMC 86k, dosage form C). The dosage forms were square disks: side length 10 mm and thickness 3 mm.

### Example 2: Scanning Electron Microscopy

The microstructures of the solid fibrous dosage forms were imaged by a Zeiss Merlin High Resolution SEM with a GEMINI column. The top surfaces were imaged after coating the sample with a 10-nm thick layer of gold. The cross-sections were imaged after the sample was cut with a thin blade (MX35 Ultra, Thermo Scientific, Waltham, MA) and coated with gold as above. The specimens were imaged with an in-lens secondary electron detector, at an accelerating voltage of 5 kV, and a probe current of 95 pA.

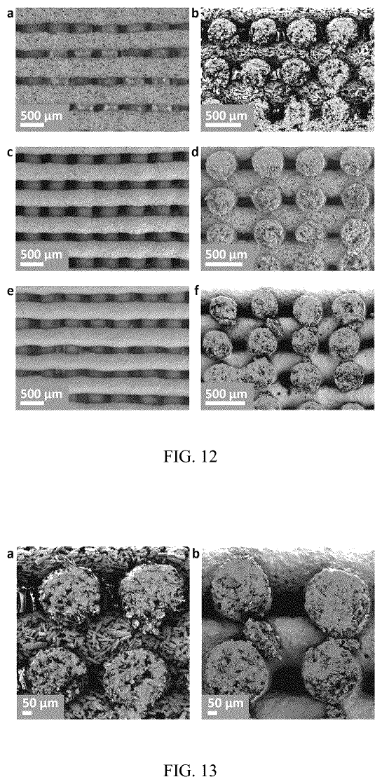

FIG. 12 presents scanning electron micrographs of the top and front views of the fibrous dosage forms. The fiber radius, R₀, in the solid dosage forms was 195, 185, and 169 μm, and the inter-fiber spacing, λ₀, in the solid dosage forms was 575, 553, and 503 μm, respectively, for the 10k, 26k, and 86k excipients, (Table 2 later). The ratio R₀/λ₄ was 0.33-0.34.

The volume fraction of the solid fibers in the dosage form may be directly estimated from the microstructural parameters and may be expressed as:

$${φ}_{s} = ξ \frac{π {R}_{0}}{2 {λ}_{0}} \qquad ( 18 )$$

where ξ is the ratio of the fiber diameter and the average thickness of a micro-patterned layer. From FIGS. 12b and 12d, ξ≈1.35. Thus, φₛ≈0.71-0.72, as listed in Table 2 later.

Moreover, the individual fibers in the structure were slightly porous. In isotropic contraction during drying the following equations may be derived to estimate the porosity in the solid fibers:

$${φ}_{pores} = 1 - {( \frac{{R}_{n}}{{R}_{0}} )}^{3} ( 1 - \frac{{c}_{solv}}{{ρ}_{solv}} ) \qquad ( 19 a )$$

$$or$$

$${φ}_{pores} = 1 - {( \frac{{λ}_{n}}{{λ}_{0}} )}^{3} ( 1 - \frac{{c}_{solv}}{{ρ}_{solv}} ) \qquad ( 19 b )$$

where Rₙ and λₙ, respectively, are the nominal fiber radius and the nominal inter-fiber spacing in the wet structure (before drying), R₀ and λ₀ are the fiber radius and inter-fiber spacing in the solid fibrous dosage form, cₛₒₗᵥ is the concentration of solvent (water) in the wet fibers, and ρₛₒₗᵥ is the density of the solvent. For the relevant parameters shown in Table 2, φₚₒᵣₑₛ≈0.11-0.12.

FIG. 13 presents magnified images of the cross sections of the dosage forms with HPMC 10k and HPMC 86k excipients to show the pores in the interior and on the surface of the fibers. The pore size (e.g., the pore diameter, or the pore width, or the pore channel width, or the pore channel height) in the fibers was about 5-50 μm for both dosage forms. The pores in the fibers were randomly (or mostly randomly) distributed.

### Example 3: Imaging Disintegrating Dosage Forms

The dosage forms were first immersed in a beaker filled with 400 ml dissolution fluid (a 0.2 M sodium phosphate buffer solution at 37° C. and a pH of 7.2). The fluid was stirred by a paddle rotating at 50 rpm. The disintegrating fibrous dosage forms were imaged from the top with a Nikon DX camera.

FIG. 14a presents images of a disintegrating dosage form with HPMC 10k excipient. Upon immersion, the dissolution fluid percolated (part of) the void space fairly rapidly. A viscous layer consisting of dispersed drug particles in an excipient-dissolution fluid solution then developed around the fibers. The layer grew inwards and outwards with time, and after 4-8 minutes the dosage form structure had entirely transitioned to a viscous suspension. The structure then fragmented and the fragments dissolved rapidly. The entire fibrous dosage form was dissolved by 10-15 minutes.

The disintegration mechanism of a dosage form with HPMC 26k excipient is shown in FIG. 14b. The disintegration mechanism was about the same as that of dosage form A; the rates of fragmentation and erosion, however, were slower.

FIG. 14c shows images of a disintegrating dosage form with HPMC 86k excipient. As in the previous cases, dissolution fluid percolated into the void space upon immersion, and the dosage form transitioned from solid to viscous. Unlike above, however, the viscous dosage form did not fragment; instead a thick viscous mass was formed that eroded very slowly. Under the experimental conditions, the erosion time was about 10 hours.

### Example 4: Drug Release

Drug release by the fibrous dosage forms was monitored using the same experimental setup and the same experimental conditions as described in Experimental Example 3 above. Additionally, an aliquot of the dissolution fluid was sampled at regular time intervals after immersion of a dosage form. The concentration of drug in an aliquot (e.g., in the dissolution fluid at a specific time after immersion of a dosage form) was determined by a Perkin Elmer Lambda 950 UV/Vis Spectrophotometer.

FIG. 15a plots the fraction of drug released by the three dosage forms versus time. The times to release eighty percent of the initial drug amount, t_{0.8}, were 11, 64, and 487 min, respectively, for the 10k, 26k, and 86k dosage forms, Table 2.

FIG. 15b shows log-log plots of the fraction of drug released versus time. The data show that the plots are essentially linear with slope about unity. Thus, the drug release rate was approximately constant.

FIG. 16 is a log-log plot of t_{0.8} versus molecular weight of the excipient, M_{w}. The t_{0.8} time increased with increasing molecular weight as t_{0.8}=0.16×M_{w}^{1.8} min.

TABLE 2
Microstructural parameters and drug release
properties of fibrous dosage forms.

| | R₀ | λ₀ | | | | t_{0.8} |
|---|---|---|---|---|---|---|
| | (μm) | (μm) | R₀/λ₀ | φₛ | φₚₒᵣₑₛ | (min) |
| A | 195 ± 8 | 575 ± 26 | 0.34 | 0.72 | 0.15 | 11 |
| B | — | — | — | — | — | 64 |
| C | 169 ± 9 | 488 ± 17 | 0.35 | 0.73 | 0.13 | 487 |

The fiber radius, R₀, and inter-fiber spacing, λ₀, are obtained from the SEM images in FIG. 1.
The volume fraction of solid fibers, φₛ = ξπR₀/2λ₀, where ξ ≈ 1.35 (FIGS. 12b and d).
φₚₒᵣₑₛ is the average from Eqs. (2a) and (2b) using R₀ and λ₀ from above, cₛₒₗᵥ = 299 (A) and 548 mg/ml (B), ρₛₒₗᵥ = 1000 mg/ml, and Rₙ = 212 and λₙ = 600 μm.
t_{0.8} values are obtained from FIG. 15a. Two samples were tested for each dosage form. The individual t_{0.8} times were 9.5 and 11.9 min (dosage form A), 60 and 68 min (dosage form B), and 419 and 555 min (dosage form C).

### Example 5: Shear Viscosity of Excipient-Water Solutions

A primary property that affects the drug release rate is the viscosity of the excipient-water solution in the fiber between the drug particles. Accordingly, the shear viscosity of excipient-water solutions was determined by a shear rheometer (TA Instruments, ARG2 Rheometer) equipped with a 60 mm diameter cone with an apex angle of 178°. The solutions consisted of water and excipient at concentrations in the range 1-20 wt % (10-200 mg/ml) for the 10 kg/mol excipient, 0.05-10 wt % (5-100 mg/ml) for the 26 kg/mol excipient, and 0.2-16.7 wt % (2-167 mg/ml) for the 86 kg/mol excipient. The temperature during the experiments was 37° C., and the shear strain-rate was 1/s.

FIG. 17 is a plot of the shear viscosity, μₛₒₗ, of excipient-water solutions versus excipient concentration, cₑ, at a shear strain rate of l/s.

Two regimes, dilute and semi-dilute, were apparent. The regimes were delineated by the disentanglement concentration, cₑ*. In the dilute regime (ce<cₑ*), the viscosity was a linear function of the excipient concentration, in agreement with the Einstein viscosity relation. In the semi-dilute regime (cₑ<cₑ*), the viscosity was a power function of the excipient concentration. The disentanglement concentrations and fit equations of the viscosity are given below.

| | | μₛₒₗ (Pa · s) | |
|---|---|---|---|
| | cₑ* | Dilute | Semi-dilute |
| Excipient | (mg/ml) | regime | regime |
| 10k | 62 | 2.5 × 10⁻⁴cₑ + 0.001 | 4.76 × 10⁻¹⁰cₑ^{4.21} |
| 26k | 20 | 6.8 × 10⁻⁴cₑ + 0.001 | 1.00 × 10⁻⁷cₑ⁴ |
| 86k | 6 | 1.8 × 10⁻³cₑ + 0.001 | 1.67 × 10⁻⁵cₑ^{.3.7} |

Dilute regime: cₑ < cₑ*; semi-dilute regime: cₑ > cₑ*.

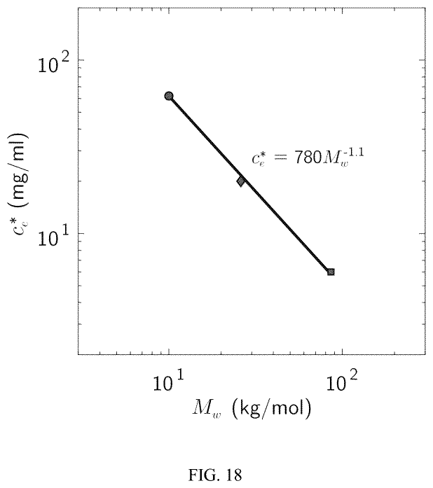

The disentanglement concentration, cₑ*, decreased with molecular weight of the excipient, M_{w}, as cₑ*=780M_{w}^{-1.1} mg/ml (tabulated above and shown in FIG. 18). The viscosity increased up to five orders of magnitude as M_{w} was increased from 10 to 86 kg/mol (tabulated above).

### Application Examples

In some embodiments, the amount of active ingredient contained in a dosage form disclosed in this invention is appropriate for administration in a therapeutic regimen that shows a statistically significant probability of achieving a predetermined therapeutic effect when administered to a relevant population. By way of example but not by way of limitation, active ingredients may be selected from the group consisting of acetaminophen, aspirin, caffeine, ibuprofen, an analgesic, an anti-inflammatory agent, an anthelmintic, anti-arrhythmic, antibiotic, anticoagulant, antidepressant, antidiabetic, antiepileptic, antihistamine, antihypertensive, antimuscarinic, antimycobacterial, antineoplastic, immunosuppressant, antihyroid, antiviral, anxiolytic and sedatives, beta-adrenoceptor blocking agents, cardiac inotropic agent, corticosteroid, cough suppressant, diuretic, dopaminergic, immunological agent, lipid regulating agent, muscle relaxant, parasympathomimetic, parathyroid, calcitonin and biphosphonates, prostaglandin, radiopharmaceutical, anti-allergic agent, sympathomimetic, thyroid agent, PDE IV inhibitor, CSBP/RK/p38 inhibitor, or a vasodilator).

In conclusion, this invention discloses a dosage form with predictable structure and drug release behaviour. Both can be tailored by well-controllable parameters. This enables improved control of the drug release and drug delivery rates into the blood stream, and thus improved control of drug concentration in blood. This further enables faster and more economical development and manufacture of pharmaceutical dosage forms, and higher quality and more personalized medical treatments.

We claim:

1. A pharmaceutical dosage form comprising:
a drug-containing solid having an outer surface and an internal structure contiguous with and terminating at said outer surface;
said internal structure comprising a three dimensional structural framework one or more repeatably arranged, fibrous elements;
said fibrous elements occupying a volume fraction of the drug-containing solid in the range of 0.45 to 0.98; and
said fibrous elements comprising an excipient matrix with drug particles and pores dispersed through their volume; wherein
the volume fraction of pores in at least one fibrous element is in the range of 0.01 to 0.4.

2. The dosage form of claim 1, wherein the three dimensional structural framework comprises a plurality of criss-crossed stacked layers of fibers.

3. The dosage form of claim 1, wherein said one or more fibers further comprise fiber segments spaced apart from adjoining segments by free spacings defining one or more free spaces through the drug-containing solid.

4. The pharmaceutical dosage form of claim 1, wherein said excipient matrix including at least one physiological fluid-soluble polymer, wherein the mass of physiological fluid-soluble polymeric excipient in the drug-containing solid divided by the volume of free space in the drug-containing solid is no greater than six times the disentanglement concentration of said physiological fluid-soluble polymeric excipient in said physiological fluid.

5. The pharmaceutical dosage form of claim 1, wherein the volume fraction or weight fraction of said dispersed drug particles in at least one fiber or fiber segment is greater than 0.5.

6. The pharmaceutical dosage form of claim 1, wherein at least a pore is filled with gas.

7. The dosage form of claim 1, wherein one or more free spaces are interconnected through the thickness of the drug-containing solid.

8. The dosage form of claim 1, wherein the effective free spacing between segments across one or more interconnected free spaces on average is in the range of 5 μm-1.5 mm.

9. The dosage form of claim 1, wherein the free spacing between segments of the one or more fibers is precisely controlled.

10. The pharmaceutical dosage form of claim 1, wherein upon immersion in a physiological fluid, said fluid percolates at least an interconnected free space, and the structural framework transitions to viscous and fragments, promoting dissolution of the active ingredient.

11. The dosage form of claim 1, wherein average thickness of the one or more fibers is in the range of 10 m to 2 mm.

12. The dosage form of claim 1, wherein at least one physiological fluid-soluble excipient is absorptive of a physiological/body fluid, and wherein rate of penetration of the physiological/body fluid into a fiber or said absorptive excipient under physiological conditions is greater than the average fiber thickness divided by 3600 seconds.

13. The dosage form of claim 1, wherein the least one physiological fluid-soluble excipient comprises a solubility greater than 5 g/l in an aqueous physiological/body fluid under physiological conditions.

14. The dosage form of claim 1, wherein at least one physiological fluid-soluble excipient is selected from the group comprising hydroxypropyl methylcellulose, hydroxyethyl cellulose, polyvinyl alcohol, polyvinylpyrrolidone, hydroxypropyl methylcellulose acetate succinate, sodium alginate, hydroxypropyl cellulose, hydroxyethyl cellulose, methyl cellulose, hydroxypropyl methyl ether cellulose, starch, chitosan, pectin, polymethacrylates (e.g., poly(methacrylic acid, ethyl acrylate) 1:1, or butylmethacrylat-(2-dimethylaminoethyl)methacrylat-methylmathacrylat-copolymer), or vinylpyrrolidone-vinyl acetate copolymer.

15. The dosage form of claim 1, wherein at least one physiological fluid-soluble excipient comprises hydroxypropyl methylcellulose.

16. The dosage form of claim 1, wherein the volume or weight fraction of physiological fluid-soluble polymer in a fiber is in the range between 0.02 and 0.35.

17. The dosage form of claim 1, wherein one or more fibers comprise at least a pore with a closed end.

18. The dosage form of claim 17, wherein upon immersion of one or more elements in a dissolution fluid a capillary pressure develops in said at least one pore with a closed end.

19. The dosage form of claim 1, wherein the pores in the fibers comprise a volume fraction in the range of 0.05 to 0.3.

20. The dosage form of claim 1, wherein the pores in the fibers comprise an average size (e.g., an average width or average diameter) in the range of 0.5 μm to 125 μm.

21. The dosage form of claim 1, wherein at least one fiber comprises a surface-connected pore.

22. The dosage form of claim 1, wherein at least one pore is filled with a matter comprising air.

23. A pharmaceutical dosage form comprising:
a drug-containing solid having an outer surface and an internal structure contiguous with and terminating at said outer surface;
said internal structure comprising a plurality of criss-crossed stacked layers of fibers;
said fibers comprising fiber segments spaced apart from adjoining fiber segments by free spacings defining one or more free spaces through the drug-containing solid, wherein the volume fraction of fibers in the drug-containing solid is in the range between 0.45 and 0.98;
said fibers further comprising an excipient matrix with drug particles and pores dispersed throughout the fiber volume;
said excipient matrix including at least one physiological fluid-soluble polymer, wherein the mass of physiological fluid-soluble polymeric excipient in the drug-containing solid divided by the volume of free space in the drug-containing solid is no greater than six times the disentanglement concentration of said physiological fluid-soluble polymeric excipient in said physiological fluid;
whereby
the volume fraction of said dispersed drug particles in at least one fiber or fiber segment is greater than 0.5;
said pores are filled with at least a gas; and
the volume fraction of pores in at least one fiber is in the range between 0.01 and 0.4.
